(19) United States
(12) Patent Application Publication
(10) Pub. No.: US 2005/0014134 A1
(43) Pub. Date: Jan. 20, 2005

(54) Viral identification by generation and detection of protein signatures

(76) Inventors: Jason Andrew Appleton West, Castro Valley, CA (US); James Frederick Stamps, Livermore, CA (US); Isaac Ramzy Shokair, Livermore, CA (US); Ronald F. Renzi, Tracy, CA (US); Victoria A. Vandernoot, Pleasanton, CA (US); Boyd J. Wiedenman, Alpharetta, GA (US); Todd William Lane, Livermore, CA (US); Julia Ann Fruetel, Livermore, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/795,549

(22) Filed: Mar. 8, 2004

Related U.S. Application Data
(60) Provisional application No. 60/452,985, filed on Mar. 6, 2003.

(51) Int. Cl. G01N 33/53; C07K 1/00; C12Q 1/70; G01L 1/20; B01D 61/58; C25B 7/00; C02F 1/469; G01F 1/64; G01N 33/564; B01D 61/42; B01D 59/42; G01N 27/26; G01L 9/18; C07K 1/26; C07K 17/00

(52) U.S. Cl. 435/5; 435/7.1; 530/350; 435/974; 435/975; 436/536

(57) ABSTRACT

The present invention provides systems and processes for the collection and identification of macromolecules, such as biologically-derived macromolecules (e.g., proteins and nucleic acids), by measuring and comparing the molecular weight signatures of macromolecular samples. Reproducible molecular weight signatures provides reliable sample identification. In the case of viruses, proteomic molecular weight signatures can be used for identifying viral agents.

### CROSS REFERENCE TO RELATED APPLICATIONS

This application claims the benefit of U.S. Provisional Application No. 60/452,985, filed Mar. 6, 2003, the entirety of which is incorporated by reference herein.

### GOVERNMENT RIGHTS

The invention was made with U.S. Government support. The Government has certain rights in the invention under DOE contract DE-AC04-94AL85000.

### FIELD OF THE INVENTION

The present invention is related to the field of micro-total analysis systems (μ-TAS). The present invention is also related to microanalytical instruments for analyzing and identifying biological substances and to methods of operating microanalytical instruments for analyzing and identifying biological substances. The present invention also relates to systems and methods for analyzing and identifying macromolecular substances and chemical compounds.

### BACKGROUND OF THE INVENTION

The field of micro-Total Analysis Systems (μ-TAS) relates to miniaturized devices where all necessary parts and methods to perform a chemical analysis are integrated. More than simply shrinking down traditional bench top techniques, μ-TAS requires innovations in many fields, including chemistry, biology, electronics, optics, materials, fluid mechanics and microfabrication.

μ-TAS devices arose as a consequence of a development when electronic circuits were miniaturized and integrated in large numbers on silicon wafers. Knowledge acquired in this process was later used in the development of microsensors (mostly physical sensors, e.g., pressure and temperature), followed by microactuators (gears, switches and motors). Developing microfluidic devices is one of the main goals of μ-TAS. Early efforts focused on micropumps and valves to manipulate fluids inside a microfabricated structure. Research in this particular area is presently intense, reflecting some of the intrinsic difficulties of realizing miniaturized devices.

In early analytical chemistry applications, μ-TAS devices performed an injection and an electrophoretic separation of a sample mixture (different fluorescent dyes), in which liquid handling was achieved with electroosmotic flow. Electrophoresis and electrokinetic fluid handling is now commonly used in many miniaturized analytical devices. μ-TAS is not limited to electrodriven separation units. A large number of components require development and integration to arrive at μ-TAS solutions for a variety of analytical problems. Many of the required functional elements for μ-TAS cover a variety of different technological disciplines, some of which include channels and fluidic connections, pumping, dosing and injecting devices, reactors, mixers, valves, physical filters, sorters, heaters, coolers, physical and chemical sensors, separation and extraction media, light sources, waveguides, detectors, optical filters, integrated electronics, electrical connections, feedback and control loops, as well as information technology to enable human perception of the events occurring at the micro scale.

The motivation and benefits of miniaturized chemical analysis systems include lower cost, reduction of sample and reagent consumption as well as waste production, high speed analyses, parallel architectures, high throughput, small footprints, compact design, reliable and simple operation, field analysis and point-of-care diagnostics, as well as integration among several units or with existing systems. Other advantages include enabling new techniques and methods as temporal and spatial dimensions are reduced.

Micro analytical systems combine micromachined microelectro-mechanical structures (MEMS) that are capable of performing sample handling and chemical separations. These systems exhibit phenomenal increases in sample discrimination over stand-alone sensors. In fact, performance of micro analytical systems is approaching that of standard laboratory analytical instruments. The development of new sensor technologies is important for addressing issues of national security applications, such as chemical/biological weapon defense. Microsensors are miniature devices that convert information about the environment into an electrical form that can be read by instruments. Sensors are increasingly used as computer input devices because of large increases in computing power and cost reductions. Microsensors thus can enable a computing machine to “sense” its environment through sight, hearing, taste, smell and touch. Accordingly, micro analytical systems promise to revolutionize a number of fields, including food processing and health care. As an example, the present inventors have been diligently working to develop a hand-held micro analytic system, identifiable as the μChemLab™ (pronounced, “micro chem lab”) system for lab on a chip chemical analysis.

At its November, 2002 annual meeting, the International Conference on Miniaturized Chemical and Biochemical Analysis Systems, μTAS, reported on the research, development and application of micro fabricated devices and systems for chemical and biochemical measurements. A number of the reported technologies include the following: Cell Growth and Monitoring; Separation; Gels for Biochemical Analysis; Micro Analysis Systems; DNA Separation; Droplet Base Fluidics; Fluid Mechanics & Design Tools; Micro Machining Methods; Micropumps & Microvalves; Clinical Diagnosis; Genomics and Proteomics; Micro-Optical Systems; DNA Assay; Nano Fluidics; Magnetic Materials; Sample Prep System; Microfluidics; Plastic Machining; Materials; Electrochemical Detection; Mass Spectrometry; Novel Detection Techniques; Environmental Assays; Separation Science; Proteomics; Sample Preparation; Detection Systems; Cell Manipulation in Flows; Separation, Concepts; Microfluidic Components; Nanotechnology; Cellular Analysis; Drug Discovery; Biochemical Applications; Novel Quantification Strategies; Single Cell Analysis; Nano-Microfabrication; and DNA Systems. Many of the details of these technologies are described further in Baba, Y. et al., eds., Micro Total Analysis Systems 2002: Proceedings of the μTAS 2002 Symposium, Nara, Japan, 3-7 Nov. 2002, Vols. 1 and 2, Kluwer Academic Publishers, Hingham, Mass., ISBN: 1-4020-1011-7, (2003), the contents of which are incorporated by reference herein in its entirety.

U.S. Pat. No. 6,475,364 discloses methods, devices and systems for characterizing proteins using capillary channels, which is incorporated by reference in its entirety. U.S. Pat. No. 5,800,690 discloses variable control of electroosmotic and/or electrophoretic forces within a fluid-containing structure via electrical forces, which is incorporated by reference in its entirety. U.S. Pat. No. 6,001,229 discloses apparatus and methods for performing microfluidic manipulations for chemical analysis, which is incorporated by reference in its entirety.

The foregoing illustrates that a great deal of research has already gone into developing devices for analyzing chemical and biological substances. However, there still remains the difficult problem of rapidly detecting and identifying viruses; For example, current methods are laborious, time consuming and depend on specific reagents for detecting single viral isoforms.

Many analytical systems for identifying viruses typically use viral-specific reagents for each known, targeted virus, such as an antibody. Antibodies are difficult to produce in mass quantities and are non-specific, particularly for viral agents. An outstanding problem is the detection of viruses using a micro analytical system that does not require the use of viral-specific reagents. In addition, immunobased (antibody based) applications take hours to days perform. Alternatively, the polymerase chain reaction (PCR) procedure is commonly-used to detect the presence of a viral agent through the genes and gene products of these organisms. PCR-based methods again suffer in that they require a specific reagent to detect a particular virus, and hence are limited in the numbers of agents that can be detected. Although these methods are more rapid than immunobased techniques, they still require as long as several hours to detect that presence of a suspected agent. Accordingly, it is desirable to detect viral agents without requiring the use of specific reagents for detecting a particular viral agent. Providing portable microanalytical systems that can be operated in the field for identifying biological, macromolecular and chemical substances is also desired.

There also remains the problem of providing sensitive, accurate detection and identification of biological and chemical weapons. New detection technologies are urgently needed to avert wide spread casualties from a bio-terrorist threat. Equally important is the ability to detect toxins at remote locations outside the controlled environment of the laboratory. The present inventions described herein provide solutions to these extremely important problems. The present invention provides for portable detectors, one embodiment of which is under development at Sandia National Laboratories and identified by the trade name “μChemLab™”. The present invention also provides for methods that are capable of discriminating among closely related toxin isoforms, such as the toxin isoforms of the biotoxin ricin.

### SUMMARY OF THE INVENTION

Certain aspects of the present invention provide for the collection and identification of macromolecules, such as biologically-derived macromolecules (e.g., proteins), by measuring the molecular weight distribution of macromolecular samples. The precision and reproducibility of the resulting molecular weight distributions afforded by this aspect of the invention enables reliable sample identification. In the case of viruses, proteomic molecular weight distributions can be used for identifying viral agents.

In various aspects of the present invention, several processes are combined for generating molecular weight distributions of viruses. Viral signatures are generated by fragmenting intact virus samples and solubilizing and fragmenting the exposed proteins on the viral protein coats. The exposed proteins are then fluorescently labeled to enable detection. The fragmented and fluorescently labeled viral proteins are electrokinetically injected into a microanalytical instrument for analysis. The labeled proteins are separated according to molecular size using microchannel separation chromatography (e.g., electrophoretic microseparation) operating in feedback control loop driven constant current mode. Detection of the labeled proteins using laser-induced fluorescence (LIF) correlates signal intensity from the LIF detector to protein concentration. The fluorescence signal intensity and microchannel separation time data are processed by a computer and correlated to viral signature information stored in a database for identifying the viral agents. Optionally, an on-chip preconcentrator is used for concentrating dilute viral samples for increasing the signal-to-noise ratio.

In related aspects of the present invention there are provided processes for identifying a virus by its proteins. In these aspects, the processes include solubilizing at least a portion of the proteins of the virus to provide solubilized proteins; providing a microfluidic chip; optionally preconcentrating the solubilized proteins on the microfluidic chip; labeling at least a portion of the solubilized proteins to provide labeled proteins on the microfluidic chip; electrokinetically injecting at least a portion of the labeled proteins into at least one microchannel electrophoretic separator on the microfluidic chip; electrophoretically separating at least a portion of the labeled proteins in a constant-current mode to provide separated proteins on the microfluidic chip; detecting at least a portion of the separated proteins on the microfluidic chip using a laser-induced fluorescence detector, wherein the detecting generates signals correlate to the concentration and separation time of the separated proteins; and analyzing the signals to identify the virus.

In further aspects of the present invention, a variety of biological and non-biological macromolecular samples can also be analyzed. In broad terms, several processes are combined in the present invention for generating molecular weight distributions of macromolecular samples. Macromolecular signatures are generated by solubilizing the macromolecular components of the samples. The macromolecular components are then fluorescently labeled to enable detection. The fragmented and fluorescently labeled macromolecules are electrokinetically injected into a microanalytical system for analysis. Detection is performed by separating the macromolecules from the macromolecular sample using microchannel separation chromatography (e.g., electrophoretic microseparation) coupled to a photon-based (e.g., light) detector. Detected light signal intensity from the detector is correlated to macromolecular concentration. The light signal intensity and microchannel separation time data are processed by a computer and correlated to macromolecular signature information stored in a database for identifying the viral agents. Optionally, an on-chip preconcentrator is used for concentrating dilute macromolecular samples.

In related aspects of the present invention there are provided processes for correlating the agent component signature to the identity of the chemical or biological agent. These processes include solubilizing components of a sample, the sample including a chemical or a biological agent to provide solubilized components; optionally preconcentrating the solubilized components; labeling at least a portion of the solubilized components with a fluorescent dye to provide labeled components; injecting the labeled components electrokinetically into at least one microchannel electrophoretic separator; separating the labeled components electrophoretically using a controlled electric field, the controlled electric field operating in a constant-current mode; detecting the separated components with a laser-induced fluorescence detector, the detector generating signals, the generated signals being correlated to the concentration and separation time of the labeled components; generating an agent component signature including the concentration and the separation time; and correlating the agent component signature to the identity of the chemical or biological agent.

In related aspects of the present invention there are provided processes for identifying a chemical agent or biological agent isoform among individual agent component signatures. These processes include solubilizing components of at least two samples including a chemical agent, a biological agent, or both, to provide solubilized components; optionally preconcentrating the solubilized components; individually labeling the solubilized components with a fluorescent dye; individually injecting the solubilized components electrokinetically into at least one microchannel electrophoretic separator; individually electrophoretically separating the labeled components using a controlled electric field operating in a constant-current mode to provide separated components; individually detecting the separated components with a laser-induced fluorescence detector, the detector capable of generating signals correlatable to the concentration and separation time of the labeled components; individually generating an agent component signature including the concentration and the separation time; and identifying a chemical agent or biological agent isoform among the individual agent component signatures.

In related aspects of the present invention there are provided processes for analyzing macromolecular signatures for use in identifying biological entities. These processes include providing a sample including macromolecules derived from a biological entity; solubilizing at least a portion of the macromolecules to provide solubilized macromolecules; optionally preconcentrating the solubilized macromolecules; labeling at least a portion of the solubilized macromolecules with a fluorescent dye to provide labeled macromolecules; electrokinetically injecting at least a portion of the labeled macromolecules into a microchannel electrophoretic separator; electrophoretically separating the labeled macromolecules using a controlled electric field operating in a constant-current mode to provide separated macromolecules; detecting the separated macromolecules using a laser-induced fluorescence detector capable of generating signals, the signals capable of being correlated to the concentration and separation time of the separated macromolecules; generating a macromolecular signature, the signature including the concentration and macromolecular separation time; and analyzing the macromolecular signature to identify the biological entity.

In further aspects of the present invention there are provided systems capable of identifying chemical and biological agents. These systems include a microfluidic chip, including; an injection port for receiving samples including protein; an optional preconcentrator; an electrokinetic pump for transporting proteins to an electrophoretic microchannel separator; and the electrophoretic microchannel separator capable of separating proteins using a controlled electric field, the controlled electric field operating in a constant-current mode; a detector giving rise to signals correlatable to the concentration and separation time of the separated proteins; and a data processor for correlating the signals to the protein signatures of known biological samples.

In other aspects of the present invention there are provided systems capable of identifying chemical and biological agents. These systems include an injection port for receiving biological samples including biological macromolecules; a microfluidic chip in fluid communication with the injection port, the microfluidic chip including: an optional preconcentrator in fluid communication with the injection port; an electrokinetic pump in fluid communication with the injection port capable of transporting the biological macromolecules to an electrophoretic microchannel separator including a controlled electric field, the controlled electric field operating in a constant-current mode; the electrophoretic microchannel separator capable of separating the biological macromolecules; a detector capable of detecting the presence of the separated biological macromolecules, the detector giving rise to signals being correlatable to the concentration and separation time of the separated biological macromolecules; and a data processor for correlating the signals to a biological macromolecular signature of a biological entity.

Further aspects of the invention provide protein detection systems capable of detecting and identifying low concentration levels of proteomic substances. These systems include: a microfluidic sample injection port capable of receiving a liquid including proteomic substances; a microfluidic chip, including: a preconcentrator in fluidic communication with the injection port, the preconcentrator including: a porous surface in fluid communication between a first channel provided in the microfluidic chip and a second channel provided in the microfluidic chip, wherein the first and second channels include deep etched portions in the microfluidic chip and a shallow etched portions in the deep etch portions, the porous surface including a cover material bonded to a rough surface, the rough surface being contiguous to the shallow etched portions; at least one microchannel capillary zone electrophoresis separator or a capillary gel electrophoresis separator in fluid communication with the preconcentrator; at least one laser-induced fluorescence detector capable of detecting the presence of proteomic substances, the detector capable of generating signals correlatable to the concentration and separation time of the proteomic substances; and a data processor capable of receiving the signals and generating a proteomic signature of a biological entity.

Other aspects of the present invention provide hand-held protein detection systems capable of detecting and identifying small amounts of proteomic substances. These systems include: at least one separation module, including: a microfluidic sample injection port capable of receiving a liquid under pressure, the liquid including proteomic substances; a fluidic system capable of electrokinetically transporting the liquid and capable of separating the proteomic substances by molecular size; and a microfluidic fluorescence detector capable of detecting the concentration and separation time of the proteomic substances; and a power supply capable of monitoring and controlling electric currents and voltages of the fluidic system, the power supply capable of generating at least one full-scale stepped voltage in at least 20 milliseconds and capable of measuring at least one current in at least 20 milliseconds.

### BRIEF DESCRIPTION OF THE DRAWINGS

FIG. 1 depicts an exploded view of a hand-held embodiment of a system of the present invention.

FIG. 2 depicts a separation module mounted on a detector.

FIG. 3 depicts an exploded view of a fluid system.

FIG. 4 depicts a view of a fluid system, view towards the compression frame with a mounted microfluidic chip and compression plate visible therethrough.

FIG. 5 depicts a high voltage power supply.

FIG. 6 depicts a high voltage board.

FIG. 7 depicts the pressure injection of a sample into an embodiment of the system of the present invention.

FIG. 8 illustrates integration of pressure and electrokinetic injection in a microfluidic system.

FIG. 9A depicts simultaneous CZE and CGE results (voltage control).

FIG. 9B depicts capillary electrophoresis results of 20 nM lactalbumin and 20 nM ovalbumin, with and without 60 second preconcentration.

FIG. 10 depicts separation of fluorescamine-labeled ricin using a microanalytical system. Bottom−300 pM ricin in standard buffer. Top−600 pM ricin in standard buffer.

FIG. 11 depicts a mask diagram for a microfluidics chip.

FIG. 12 depicts the microchannel flow in a microfluidics chip in both injection and separation modes.

FIG. 13 depicts virus analyses using one embodiment of a system of the present invention.

FIG. 14 illustrates improvements made using constant current mode versus constant voltage mode.

FIG. 15 illustrates reproducibility results of phage T2 under constant current mode.

FIG. 16 illustrates generation of mass signatures.

FIG. 17A depicts retention time corrected chromatograms for species (Bacteriophage T2) for two measurements.

FIG. 17B depicts retention time corrected chromatograms for species (Bacteriophage T4) for two different measurements.

FIG. 17C compares corrected chromatograms for species (Bacteriophage T2) and (Bacteriophage T4).

FIG. 18A depicts a software diagram, and FIG. 18B depicts a hardware diagram for a current control mode.

FIG. 19 depicts one embodiment of a microfluidic chip including a preconcentrator. Inset shows a close-up view of the preconcentrator.

FIG. 20A illustrates a cross section of the preconcentrator in FIG. 19 looking along direction I.

FIG. 20B illustrates a cross section of an alternate embodiment of a preconcentrator that includes a shallow-etched regions in contact with the narrow gap.

### DETAILED DESCRIPTION OF ILLUSTRATIVE EMBODIMENTS

Various embodiments of the present invention provide systems and methods for identifying the identity of various biological entities, such as a virus or biotoxin. The systems of the present invention are preferably hand-held or otherwise able to be transported to a site for sample collection. Such portable systems are useful, for example, for use by a first-responder such as a fireman, policeman, medical worker, or the like in determining the presence of a biotoxin or other threat. Generally, in embodiments of the invention, a sample is injected into the system. A microfluidic separation is typically performed and at least one separated component is typically detected by a detector module within the systems of the present invention. A target analyte is identified, based on the separated component, and the presence of the target analyte is indicated on an output interface, such as a display, of the systems, in accordance with embodiments of the invention.

In some embodiments, a plurality of separations are performed on the sample to enhance or verify the identification of the target analyte. In some embodiments, as will be further described below, dilute analyte samples can be preconcentrated on the microfluidic chip to improve detection of minute quantities of biomolecular samples. In some embodiments, the systems are modular and various components—including, for example, the detector module—can be removed and replaced between separations. Further, in some embodiments a plurality of samples can be analyzed sequentially, and/or simultaneously with the previous samples being stored in a waste reservoir, as is described further below. In some embodiments, the microfluidic separation is performed in a separation channel using constant-current control. Generally, as described further below, constant-current control improves separation performance, for example, by improving run-to-run reproducibility to enhance detection. In some embodiments, one or more reservoirs are in fluid communication with a microfluidic chip within the device through a fluid manifold base. This allows one or more reservoirs to be removed and replaced without introducing gas to the microfluidic chip. A general description of a device having subsystems useful with embodiments of the present invention is also found in G. A. Thomas, et. al. “μChemLab™—An Integrated Microanalytical System for Chemical Analysis Using Parallel Gas and Liquid Phase Microseparations”, Proc. SPIE Vol. 3713, p. 66-76, Unattended Ground Sensor Technologies and Applications, Edward M. Carapezza; David B. Law; K. Terry Stalker; Eds., July 1999, hereby incorporated by reference in its entirety.

Accordingly, the present invention provides methods and devices for determining the presence of a target analyte. By “target analyte” or “analyte” or grammatical equivalents herein is meant any molecule or compound to be detected, defined below. Generally any target analyte that is detectable using the separation methods described further below may be used.

As used herein, the terms “biomolecule” and “biological molecule” are used interchangeably. Suitable biomolecules include, but are not limited to, proteins (including enzymes, immunoglobulins and glycoproteins), nucleic acids, lipids, lectins, carbohydrates, hormones, whole cells (including procaryotic (such as pathogenic bacteria) and eucaryotic cells, including mammalian tumor cells), viruses, spores, etc. Preferred biological molecule analytes that can be detected according to the present invention include proteins, amino acids, polysaccharides, nucleic acids, as well as fragments and combinations thereof.

Suitable liquid samples may also include small chemical molecules such as environmental, clinical chemicals, pollutants, toxins (e.g. sarin), and small biomolecules, including, but not limited to, pesticides, insecticides, toxins (including biotoxins), therapeutic and abused drugs, hormones, antibiotics, antibodies, organic materials, etc.

In preferred embodiments, the liquid sample comprises a biotoxin. As will be appreciated by those in the art, there are a large number of possible biotoxins that may be identified using embodiments of the present invention, including, but not limited to, ricin, botulinum toxin, tetanus toxin, cholera toxin, abrin, aflotoxins, and conotoxins.

In preferred embodiments, the liquid sample comprises a weapon degradation product. Degradation products that may be identified using embodiments of the present invention include, but are not limited to, alkylphosphonic acids and related monoesters.

In preferred embodiments, the liquid sample comprises an explosive. Explosives that may be identified using embodiments of the present invention include, but are not limited to, RDX, HMX, tetryl, trinitrotoluene, other nitrotoluenes and nitroaramines.

In a preferred embodiments, the liquid sample comprises a protein. As will be appreciated by those in the art, there are a large number of possible proteinaceous target analytes that may be detected using the present invention. By “proteins” or grammatical equivalents herein is meant proteins, oligopeptides and peptides, derivatives and analogs, including proteins containing non-naturally occurring amino acids and amino acid analogs, and peptidomimetic structures.

Suitable protein target analytes include, but are not limited to, immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses or bacteria outlined below.

Various embodiments of the present invention provide systems that include fluid handling mechanisms for sample preparation and separation, one or more microfluidic chips for sample transport and separation, one or more detectors for detecting sample analytes and one or more data processors for analyzing signals and identifying the analytes. Related systems, components, and their operation are described, for example, in U.S. patent application No. 10/633,871, entitled “Portable Apparatus for Separating Sample and Detecting Target Analytes”, filed Aug. 4, 2003, the entirety of which is incorporated by reference herein. The design and operation of various system components useful in the present invention, such as reservoir modules, injectors, microfluidic chips, are further described in, for example, U.S. patent application Ser. No. ______ filed 2 Apr. 2003, entitled “Micromanifold Assembly”, Docket No. SD-8367, U.S. patent application Ser. No. ______, filed 2 Apr. 2003 entitled “High Pressure Capillary Connector,” Docket. No. SD-8357, U.S. patent application Ser. No. ______, entitled “Fluid Injection Microvalve,” filed 24 Jan. 2003, Docket. No. SD-8369, U.S. patent application Ser. No. ______, filed 27 Jan. 2003 entitled “Microvalve,” Docket No. SD-8368, U.S. patent application Ser. No. ______, filed 24 Jan. 2003 entitled “Capillary Interconnect Device,” Docket No. SD-8365, U.S. patent application No. 10/350,628, entitled “Edge Compression Manifold Apparatus,” filed 24 Jan. 2003, U.S. patent application No. 10/633,871, entitled “Portable Apparatus for Separating Sample and Detecting Target Analytes”, filed Aug. 4, 2003, and U.S. Pat. No. 6,290,909, entitled “Sample Injector for High Pressure Liquid Chromatography”, all of which are hereby incorporated by reference in their entirety. Microfluidic chips and their operation are further described in International Application No. PCT/US00/30422, “Microfluidic Devices with Thick-Film Electrochemical Detection”, filed Nov. 3, 2000, the entirety of which is incorporated by reference herein.

Suitable biomolecules that can be detected according to the methods and systems of the present invention may originate from on or more biological entities, examples including but not limited to: (1) viruses, including but not limited to, orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picomaviruses, and the like; and (2) bacteria, including but not limited to, a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. Y. pestis, Pseudomonas, e.g. P. aeruginosa, P. putida; Chlamydia, e.g. C. trachomatis; Bordetella, e.g. B. pertussis; Treponema, e.g. T. palladium; and the like.

Other suitable biomolecules that can be detected include, but are not limited to, (1) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphotase; aldolase, prostatic acid phosphatase, terminal deoxynucleotidyl transferase, and bacterial and viral enzymes such as HIV protease; (2) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors, human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone and testosterone; and (3) other proteins (including fetoprotein, carcinoembryonic antigen CEA, cancer markers, etc.).

Certain embodiments of the present invention identify the origins of one or more biomolecules in a fluid sample. As will be appreciated by those in the art, the sample fluid may comprise any number of things, including, but not limited to, bodily fluids (including, but not limited to, blood, urine, serum, lymph, saliva, anal and vaginal secretions, perspiration and semen; and solid tissues, including liver, spleen, bone marrow, lung, muscle, brain, etc.) of virtually any organism, including mammalian samples; environmental samples (including, but not limited to, air, agricultural, water and soil samples); biological warfare agent samples; research samples (e.g., in the case of nucleic acids, the sample may be the products of an amplification reaction; or in the case of biotoxins, control samples, for instance; purified samples, such as purified genomic DNA, RNA, proteins, etc.; raw samples (bacteria, virus, genomic DNA, etc.). As will be appreciated by those in the art, virtually any experimental manipulation may have been done on the sample prior to its use in embodiments of the present invention. For example, a variety of manipulations may be performed to generate a liquid sample of sufficient quantity from a raw sample. In some embodiments, gas samples and aerosol samples are passed through a collector to generate a liquid sample containing target analytes present in the original sample. In this manner, environmental sampling of gas and/or aerosols may be used. In some embodiments, a liquid may be contacted with a solid sample to disperse the target analyte into the liquid for subsequent analysis.

Certain embodiments of the invention provide portable systems and methods for detecting a target analyte using a portable system. By ‘portable’ herein is meant that the system—including a microfluidic chip, detector module, and power supply, as described further below, for performing one or more microfluidic separations—is able to be transported to a site of sample collection. Accordingly, the microfluidic separation is able to be performed at the site of sample collection—a battle field, an accident scene, a doctor's office, an ambulance, or any other location where a sample is collected. In some embodiments, however, the system is portable, but remains located in a central location and samples are brought to the system. In some embodiments, the system is temporarily or permanently affixed to a non-portable structure, such as a wall, pipe, tank, building, office, factory, stadium or other structure, etc. and samples are brought to or taken by the system. For example, the system may be used as a detector in communication with a sensing module, as is described in U.S. patent application Ser. No. 10/402,383, filed 28 Mar., 2003 entitled “Systems and Methods for Detecting and Processing”, hereby incorporated by reference in its entirety. The output interface of the system may be coupled to the sensing module, in one embodiment. Suitable sensing modules typically include a processor and a wireless modem. The sensing unit may be in communication with one or more other sensing units for communicating data collected by one or more embodiments of portable systems according to the present invention. In some embodiments, the sensing module is configured to model data to be generated by an embodiment of the portable system according to the present invention.

In preferred embodiments, the system is hand-held, that is able to be carried by a single individual, preferably by hand, to a location. Accordingly, in preferred embodiments, the portable system weighs between about one and about ten pounds, more preferably between about three and about four pounds. In preferred embodiments, the portable system is between about 500 and about 3,000 cubic centimeters in volume, more preferably between about 500 and about 1,500 cubic centimeters, and most preferably about 1 liter in volume. This facilitates use, for example, by first-responders such as firemen, policemen, and medical workers. In other embodiments, the system is carried in a backpack, belt strap, suitcase or other personal carrying system. In other embodiments, the portable system is transported by a vehicle. The portable system has an input port and an output interface. In some embodiments the input port and output interface are not contained within the same housing. For example, in some embodiments the modules needed to perform a microfluidic separation—including an inlet, microfluidic chip, detector module, power supply, and reservoir module, as described further below—are positioned in a sample collection location, and may be mobile (for example, by autonomous or remote control)—for example, taking a sample in an location inaccessible or dangerous for a person or other reader of the output interface, and sending data relating to the microfluidic separation to an output interface in a different location. The output interface and modules needed to perform microfluidic separation, in such embodiments, are in communication via electronic, optical, or wireless means, as known in the art. In preferred embodiments, the system is self-powered, for example, by batteries, as known in the art.

Certain embodiments of the system according to the present invention comprise various components, such as a sample introduction port, a reservoir module, a microfluidic chip, a power supply, a detector, CPU controller or other processor and or/control software, and an output interface. In some embodiments, one or more of those components are not present. In accordance with some embodiments, the interfaces between these components are typically standardized, such that individual components can be disconnected, or removed from their position and replaced with other suitable components.

For example, as described further below, in some embodiments, the microfluidic chip includes a plurality of inlets, as described further below, for fluid communication with one or more reservoirs and/or sample introduction ports. The fluidic portion of the separation module, also referred to herein as a reservoir module, as described further below, comprises a plurality of reservoirs. The reservoir module, as described further below, typically includes a plurality of reservoirs coupled to a fluid manifold base, which in turn is coupled to a microfluidic chip. The reservoirs may be coupled to the fluid manifold base in such a way that one or more reservoirs may be removed and replaced (with the same or a different reservoir) without introducing gas, e.g. a bubble, into the microfluidic chip. In some embodiments, each reservoir comprises a seal and the reservoir module comprises one or a plurality of needles, each piercing the seal of a reservoir to facilitate fluidic communication with inlets on the microfluidic chip, in accordance with embodiments of the present invention. In some embodiments, one or more of the reservoirs are provided with an electrode for electrical communication between the power supply and the contents of the reservoir. The detector is positioned and configured to detect one or more target analytes on or in the microfluidic chip, as described further below. A power supply is provided in electronic communication with the reservoir module, microfluidic chip, and/or detector, as needed, in embodiments of the invention. The detector is positioned to detect target analytes in or on the microfluidic chip. For example, in one embodiment the detector includes an optical source and is positioned such that the light source illuminates a detection area on the microfluidic chip and illumination from the microfluidic chip is received by the detector. The detector may be in further communication with the power supply, as needed. Some embodiments of the invention include a processor and user interface, as described further below. The processor is in communication with the power supply, detector, and/or output interface as needed in embodiments of the invention.

In some embodiments, the microfluidic chip includes a plurality of inlets, as described further below, for fluid communication with one or more reservoirs and/or sample introduction ports. The reservoir module, as described further below, comprises a plurality of reservoirs. The reservoir module, as described further below, includes a plurality of reservoirs coupled to a fluid manifold base, which in turn is coupled to a microfluidic chip. The reservoirs are coupled to the fluid manifold base in such a way that one or more reservoirs may be removed and replaced (with the same or a different reservoir) without introducing gas, e.g. a bubble, into the microfluidic chip. In some embodiments, each reservoir comprises a seal and the reservoir module comprises one or a plurality of needles, each piercing the seal of a reservoir to facilitate fluidic communication with inlets on the microfluidic chip, in accordance with embodiments of the present invention. In some embodiments, one or more of the reservoirs are provided with an electrode for electrical communication between the power supply and the contents of the reservoir. The detector module is positioned and configured to detect one or more target analytes on or in the microfluidic chip, as described further below. The power supply is in electronic communication with the reservoir module, microfluidic chip, and/or detector module, as needed, in embodiments of the invention. The detector module is positioned to detect target analytes in or on the microfluidic chip. For example, in one embodiment the detector module includes an optical source and is positioned such that the light source illuminates a detection area on the microfluidic chip and illumination from the microfluidic chip is received by the detector module. The detector module may be in further communication with the power supply, as needed. Some embodiments of the invention include a processor and user interface, as described further below. The processor is in communication with the power supply, detector module, and/or output interface as needed in embodiments of the invention.

The interconnected modules are, in some embodiments, preferably placed into a single housing, as described further below. The modules are preferably positioned in the housing such that they are removable. For example, in some embodiments the power supply is affixed to a processor board, and installed into the housing. In one embodiment, the detector module is affixed to the housing through a dove-tail rail, or other removable mechanism, as known in the art. The reservoir module is mounted above the detector module, in one embodiment. As described further below, it is to be understood that any number of physical methods of integration of the modules may be used—mechanical screws, flanges, rails, slots, connectors, and the like—while maintaining features of one or more of the modules that allow the integration.

In embodiments of the present invention, the reservoir module comprises a plurality of reservoirs. Any number of reservoirs may generally be provided, and the number will vary based on the application, the size of the reservoirs, the desired size of the resultant device, the chip being used, and the like. In one embodiment, between 1 and 10 reservoirs are provided, although a fewer or greater number of reservoirs may also be used. The reservoirs each contain a fluid and a seal. In a preferred embodiment, the reservoir contains a macroscopic amount of fluid—that is, greater than 20 μL of fluid. In some embodiments, each reservoir is configured to contain between 20-5000 μL of fluid, more preferably between 100 and 1,000 μL, more preferably between 200-500 μL. Of course, in some embodiments a reservoir configured to contain a greater or smaller amount of fluid may be provided. In some embodiments, the reservoir module includes one or more reservoirs configured to contain a microscopic amount of fluid—such as an amount of fluid less than 20 μL. However, it is desirable that the reservoir module itself be macroscopic such that a user could manipulate, remove, and/or replace the reservoir module by hand, or by a robotic system.

In some embodiments, a reservoir comprises one or more chambers. In some embodiments, the chambers are in fluidic communication, however, in some embodiments fluids are confined to the individual chambers. In some embodiments, the chambers are in electronic communication, however, in some embodiments the individual chambers are electronically isolated. In a preferred embodiment, a reservoir comprises two chambers separated by a barrier, such as, for example, an ion permeable membrane, salt bridge, dialysis membrane, polymer film, diffusion membrane, ionomer, e.g. Nafion from Dupont, nanoporous glass, e.g. Vycor from Corning, and/or the like. In some embodiments, one chamber contains a fluid to be contacted with the microfluidic chip. A second chamber contains a fluid in contact with an electrode and is not in fluid communication with the microfluidic chip. The barrier permits electrical communication between the two chambers, in this embodiment, and prevents fluidic communication between the chambers. In this manner, fluid entering the microfluidic chip is not altered by any effects of applying a voltage across the fluid, such as pH change.

The particular fluid contained by a reservoir varies according to the application contemplated. Reservoirs generally contain reagents desired for use on a microfluidic chip, including but not limited to salts, buffers, neutral proteins (e.g. albumin), detergents, water, organic liquids with one or more components, polymers, surfactants, etc. which may be used to facilitate optimal reaction conditions and/or detection conditions, as well as optionally reduce non-specific or background interactions. Also reagents that otherwise improve the efficiency of the assay, such as protease inhibitors, nuclease inhibitors, anti-microbial agents, etc., may be used, depending on the sample preparation methods and purity of the target. Reservoirs may also contain separation media, as described further below. Preferred reagents for positioning in the reservoirs include, but are not limited to borate buffer, carbonate buffer, phosphate buffer, Tris buffer, phytic acid buffer, protein or DNA sieving gels, additives such as SDS, and other surfactants.

Generally, one or more reservoirs are coupled to the microfluidic chip by the fluid manifold base, described above. In some embodiments, a reservoir comprises a connector such as a hollow threaded connector, a tube with a gasket or o-ring, and the like. The fluid manifold base comprises a complimentary structure for the structure on the exterior of the reservoir. The fluid manifold base transports fluid from the reservoir through the structure to the microfluidic chip. The connector on the reservoir may or may not freely evolve fluid when not connected to the fluid manifold base. The connector on the reservoir or the complimentary structure on the fluid manifold base can comprise a valve and/or a seal, in some embodiments. The connector and/or complimentary structure may comprise one or more individual components comprised of the same or different materials. When a reservoir and the fluid manifold base are mated the connection provides a leak-free, contiguous fluid communication between the reservoir and the microfluidic chip. For example, in some embodiments, the structure on the bottom surface of the reservoir comprises a needle as described above. The complimentary structure attached to the microfluidic chip comprises an interface that is comprised of a seal in similitude to the seal for the bottom of the reservoir, described below. A leak resistant, contiguous fluid communication between the reservoir and the fluidic chip is formed by piercing the seal of the microfluidic chip with the needle connection of the reservoir. For example, in some embodiments, the structure on the bottom surface of the reservoir is a hollow, threaded mechanical fitting. The complimentary structure attached to the microfluidic is a hollow connector for mating to the threaded fitting on the reservoir. A leak resistant, contiguous fluid communication is formed by screwing the reservoir into the microfluidic chip.

In some embodiments, each reservoir within the reservoir module contains a seal for interface with the microfluidic chip, described below. The seal prevents or minimizes evaporation of the fluid in the reservoir and prevents leakage or spillage of fluid from the reservoir during operation and/or during removal of the reservoir or of the reservoir module from the apparatus, or during the assay. However, in most embodiments, the seal also allows penetration of a needle into the individual reservoir. Accordingly, the seal can be a cap, lid, polymer, membrane, bipolymer membrane, septum, thin film polymer, etc. Alternatively, the seal may comprise multiple components, for example a flexible polymer through which a needle will go, attached to the reservoir vial with an adhesive. In some embodiments, the seal comprises a valve, as described further below.

Reservoirs may be made from any of a number of materials, including, but not limited to, Teflon, polyetheretherketone, polyfluoroethylene, polyoxymethylene, polyimide, polyetherimide, other polymer materials, glass, fused silica and/or ceramic. Preferred materials for reservoir construction are transparent or semitransparent, in order to be able to view the fluid levels in the reservoirs. Preferred materials further have low conductivity and high chemical resistance to buffer solutions and/or mild organics used for separation media.

In some embodiments, the reservoir module includes a reservoir base which defines at least one depression or hole defining a reservoir, or into which a reservoir may be placed. For example, the reservoir base may be configured to receive a plurality of different reservoirs, such as vials, comprising reagents and/or sample or other fluid. In embodiments of the invention, the holder and the vials may be made of the same or different materials. Reservoir base materials may include, but are not limited to, the same materials used for reservoirs, described above, other machinable or moldable polymeric materials, insulators, ceramics, metals or insulator-coated metals. In a preferred embodiments, the reservoir and reservoir base materials are constructed from a polymer material that is resistant to alkaline aqueous solutions and mild organics.

In some embodiments, the reservoir base of the reservoir module defines at least one reservoir, that is, the reservoir and reservoir base are one contiguous piece. Accordingly, in some embodiments the fluid in the reservoirs is in direct contact with the reservoir base, and in other embodiments the fluid is contained in another reservoir that is placed into the reservoir base. Generally, any material suitably mechanically stable for defining at least one reservoir or depression and holding the reservoirs such that the needles in the fluid manifold base and/or on the microfluidic chip may be inserted into the reservoirs, may be used.

Embodiments of the reservoir module further include a fluid manifold, comprising a fluid manifold base and a compression plate, in embodiments of the invention. In some embodiments, the fluid manifold is continuous with the reservoir base, and in other embodiments, the fluid manifold is a separate component, formed from a same or different material as the reservoir base, and affixed to the reservoir base with adhesives and/or mechanical means, for example screws or magnets. The fluid manifold, in some embodiments, comprises a fluid manifold base defining a plurality of depressions with which the reservoirs, as defined by the reservoir base, are in fluid communication. In some embodiments, the reservoirs are inserted into the reservoir base, and further protrude into the fluid manifold. In other embodiments, the depressions defined by the reservoir base are contiguous with depressions defined by the fluid manifold base. The depressions in the fluid manifold base may be smaller, larger, or the same size as depressions defined by the reservoir base. The fluid manifold base generally serves to provide a leak-free (or low-leakage), electrically resistive, confinement pathway for liquid to traverse from one or more reservoirs to the microfluidic chip.

As described above, in some embodiments the fluid manifold base includes complementary structures to those found on one or more reservoirs, to facilitate mating the reservoirs to the fluid manifold base. In some embodiments, the fluid manifold base contains a needle that penetrates a seal of a reservoir placed in the fluid manifold base. The needle can generally be any sturdy tube, or other hollow cross-section, that can pass fluid from the reservoir to the microfluidic chip. It can be blunt, rounded, or sharp tipped. The tip may have a convex or concave shape, in accordance with embodiments of the present invention. The mechanical properties of the exposed tip of the needle have sufficient hardness and sharpness to penetrate the seal on the reservoir (or enter a pierced hole on the reservoir seal) without breaking. The needle allows a contiguous fluid stream to pass from the reservoir to the microfluidic chip, in some embodiments. Accordingly, the particular diameter of the needle may vary according to the particular fluid and application contemplated. The needle may be made of metals, polymers, glass, ceramics, semiconductors, or the like, as known in the art. The needle material may be modified from the original material to provide a more reliable connection through the reservoir seal. For example, the outside surface of a capillary may be chemically modified to increase the surface tension of the capillary/reservoir seal interface to reduce the rate of leakage, in some embodiments. The material and dimensions of the needle are chosen so that the needle maintains a contiguous fluid stream after the reservoir is reset (or removed). That is, the needle prevents air incursion, such as a bubble, into the microfluidic chip when removing and/or replacing a reservoir or the entire reservoir base. In a preferred embodiment, one or more needles are made of fused silica coated with polyimide. The needle is positioned such that it is in fluidic communication with one or more inlets of the microfluidic chip, as described further below. Further, the presence of the needle allows for a reservoir to be removed and replaced without the introduction of a bubble into the fluid stream.

Needles may include one or more components, in accordance with embodiments of the invention. The individual components are made from the same or different materials. One or more of the individual components pierces the seal of the reservoir. One or more of the components is involved with creating a leak resistant interface between the needle and the seal of the reservoir. The individual components of the needle are connected by mechanical, physical and/or chemical means including, but not limited to, adhesives such as epoxies or glues, melting, welding, soldering, clamping, compressing or fusing. The individual components of the needle are connected to one or more of the individual components of the needle.

In preferred embodiments, polymer fittings are used to attach the needle to the chip and/or to the fluid manifold base. Fittings suitable for use with the present invention are described further, for example in U.S. patent application Ser. No. ______ filed 2 Apr. 2003, entitled “Micromanifold Assembly”, Docket No. SD-8367, U.S. patent application Ser. No. ______ filed 2 Apr. 2003 entitled “High Pressure Capillary Connector,” Docket. No. SD-8357, U.S. patent application Ser. No. ______ entitled “Fluid Injection Microvalve,” filed 24 Jan. 2003, Docket. No. SD-8369, U.S. patent application Ser. No. ______ filed 27 Jan. 2003 entitled “Microvalve,” Docket No. SD-8368, U.S. patent application Ser. No. ______ filed 24 Jan. 2003 entitled “Capillary Interconnect Device,” Docket No. SD-8365, and U.S. patent application Ser. No. 10/350,628, filed 24 Jan. 2003, all of which are hereby incorporated by reference in their entirety. In one embodiment, however, the needle is attached directly to an inlet of the microfluidic chip with an adhesive, for example.

Needles can be attached to the microfluidic chip directly or indirectly. In some embodiments, the needle is attached directly to the inlet of a microfluidic chip by forming a leak resistant seal between the needle and an inlet of the microfluidic chip such that the needle can transport fluid into the microfluidic chip. Methods for attaching the needle to the microfluidic chip include, but are not limited to, adhesives such as epoxies and glues, melting, welding, soldering or fusing. In some embodiments, the needle and inlet of the microfluidic chip are mated via a separate connection. For example, in one embodiment, the connector on the needle can be separate from or contiguous to or attached to the material comprising the needle. A connector complimentary to the needle connector is found attached to the microfluidic chip. The connector and the complimentary connector are comprised of one or more individual components and are comprised from the same or different materials. The complimentary connector to the fitting attached to the needle is machined into the fluidic manifold base, in some embodiments. For example, the needle may be in a fitting that is screwed into the fluid manifold base. The needle and needle connection screw into the complimentary connection in the fluid manifold base and the needle fitting compresses against the needle forming the leak resistant seal.

The fluid manifold base is coupled to a microfluidic chip, as described further below, using a compression plate. The compression plate is configured to support the microfluidic chip and compress it against the fluid manifold base, using structures known in the art, such as screws, clamps, clips, vises, magnets, solenoids, etc. Seals such as o-rings or gaskets are placed between the microfluidic chip and the fluid manifold base such that the microfluidic chip is in fluid communication with one or more needles via one or more input ports on the microfluidic chip, as described further below. The o-rings or gaskets may be formed from any number of materials, including, but not limited to, rubber, silicone, Viton, Buna-N, Teflon, nitrile, neoprene, polyurethane, EPDM, perfluoroelastomer, fluorosilicone, etc. The particular o-ring material chosen is dependent on the chemical resistance of the material to the liquid media used, in some embodiments. In embodiments using o-rings, the o-rings are positioned around one or more input ports of the microfluidic device such that fluidic communication is established between the microfluidic device and a reservoir. The compression plate generally provides a surface for an evenly distributed compression force to be applied to the microfluidic chip for sealing against the o-rings or gaskets. The compression plate can be made form any material with sufficient toughness to withstand the compression forces required to hold the chip against the o-rings—including, for example, glass, polymer, metal, semiconductor, insulator, ceramic, and the like.

In some embodiments, the compression plate comprises one or more separate parts used to compress the microfluidic chip against the fluid manifold base. In embodiments where the compression plate comprises more than one separate part, the individual parts can be made from the same or different materials. The compression plate can be opaque, semitransparent or transparent. In preferred embodiments, one part of the compression plate is transparent, such as glass. For example, the compression plate can be made from two parts—a metal frame and a glass plate, in some embodiments. In some embodiments, the compression plate contains mechanical features, such as depressions, tabs, or holes to accommodate the modular nature of the device. In a preferred embodiment, the compression plate contains a hole that allows access to the separation channel of the microfluidic chip by the detector module. In a preferred embodiment, the compression plate includes depressions for metal pins used for alignment of the reservoir module to the detector module.

In some embodiments, as described further below, the reservoir module further comprises an introduction port. In some embodiments, the introduction port is the sample introduction port for the portable device. In other embodiments, the fluid manifold introduction port is in fluid communication with a sample introduction port in an external housing, through tubing, channels, or other means known in the art, for example when a gaseous or aerosol sample is being collected. In some embodiments, a plurality of introduction ports are provided. The introduction port, in some embodiments, is placed on one side of the fluid manifold base. One or more channels are provided in the fluid manifold base coupling the introduction port to one or more input ports on the microfluidic device. In preferred embodiments, the channel is as short as can be formed in the fluid manifold base to minimize the amount of fluid necessary to fill the channel. In a preferred embodiment, an injector port has a sample injector volume between the port and the microfluidic chip of less than 1500 nanoliters, more preferably less than 1000 nanoliters, still more preferably less than 500 nanoliters, and most preferably about 50-100 nanoliters. In a preferred embodiment, for example, the sample fluid containing a target analyte of interest is injected into the microfluidic device through the introduction port in the fluid manifold base. Embodiments of the introduction port can accommodate, for example, a standard syringe, tubing, pumps such as electrokinetic pumps, peristaltic pumps, hydrostatic pumps, displacement pumps, balloon, bladder, or any other injection mechanism as known in the art—including simply contacting the introduction port with a sample, such as by spitting. In some embodiments, an introduction port is provided in fluidic communication with a channel in the fluid manifold base that connects to one or more reservoirs in the reservoir module. Accordingly, in preferred embodiments, one or more reservoirs may be filled, refilled, or added to by injecting fluid into an introduction port.

Further, in preferred embodiments, one or more reservoirs in the reservoir module contain an electrode for interconnection to the power supply, described further below. The electrode is positioned to be in contact with the fluid contents of one or more reservoirs. In this manner, fluid may be transported within the microfluidic chip by the application of voltages to one or more electrodes in reservoirs, in accordance with embodiments of the invention. For example, in some embodiments, a contiguous fluid stream exists between two reservoirs through one or more channels in the microfluidic device. By applying a voltage or current between the two reservoirs, fluid is transported toward one of the reservoirs, determined by the polarity of the voltage or current application and the fluid used.

Accordingly, an electrode may be positioned in or on a reservoir in generally any way allowing electrical contact with the fluid contents and the power supply. In some embodiments, the electrode is affixed to the reservoir base, and extends into the reservoir. In some embodiments, the electrode is affixed to the fluid manifold base, and extends into the reservoir. In some embodiments one or more needles, as described above, serve as an electrode in contact with the reservoir fluid. In one embodiment, where a reservoir is provided that is placed into the reservoir base, the electrode is provided on a reservoir cap that is mechanically coupled to the reservoir. For example, a reservoir cap may screw onto, or fit over the top of a reservoir that is placed into the reservoir base. The reservoir cap comprises an electrode extending into the reservoir and an interconnect accessible from the outside of the cap. The cap may be formed from a variety of materials, preferably insulating materials. However, in some embodiments the cap is a conductive material and the entire cap forms an electrode, with a portion extending into the reservoir. The electrode may be formed from any of a variety of conductive materials including metals such as gold, tungsten, aluminum, platinum, porous carbon, and the like. In preferred embodiments, the electrode material is chosen such that any reaction between the electrode and the fluid in the reservoir is minimized.

The composition of the reservoir module (either the whole block or the base and the vials), can be made of a wide variety of materials, generally the same materials that comprise the chip, described below. In general, any material can be used, with materials that are in direct contact with the fluids in the reservoirs being preferably chemically inert. Suitable materials include glass and modified or functionalized glass, fiberglass, ceramics, mica, plastic (including acrylics, polystyrene and copolymers of styrene and other materials, polypropylene, polyethylene, polybutylene, polyimide, polycarbonate, polyurethanes, halogenated plastics, e.g., Teflon™, and derivatives thereof, etc.), GETEK (a blend of polypropylene oxide and fiberglass), polysaccharides, nylon or nitrocellulose, resins, silica or silica-based materials including silicon and modified silicon, carbon, inorganic glasses and a variety of other polymers, as also described above. The use of conductive-materials has limited application, in some embodiments.

By ‘microfluidic chip’ herein is generally meant a substrate configured for handling small amounts of fluid, generally nanoliters, although in some applications a larger or smaller fluid volume will be necessary. Microfluidic chips are typically constructed substantially of a substrate. The substrate can be made of a wide variety of materials and can be configured in a large number of ways, as is discussed herein and will be apparent to one of skill in the art. The composition of the substrate will depend on a variety of factors, including the techniques used to create the device, the use of the device, the composition of the sample, the analyte to be detected, the size of internal structures, the presence or absence of electronic components, and the technique used to move fluid, etc. Generally, the devices of the invention are easily sterilizable, although in some applications this is not required. The devices could be disposable or re-usable.

In certain embodiments, the substrate can be made from a wide variety of materials including, but not limited to, silicon, silicon dioxide, silicon nitride, glass and fused silica, gallium arsenide, indium phosphide, III-V materials, PDMS, silicone rubber, aluminum, ceramics, polyimide, quartz, plastics, resins and polymers including polymethylmethacrylate, acrylics, polyethylene, polyethylene terepthalate, polycarbonate, polystyrene and other styrene copolymers, polypropylene, polytetrafluoroethylene, superalloys, zircaloy, steel, gold, silver, copper, tungsten, molybdeunm, tantalum, KOVAR, KEVLAR, KAPTON, MYLAR, teflon, brass, sapphire, etc. High quality glasses such as high melting borosilicate or fused silicas may be preferred for their UV transmission properties when any of the sample manipulation and/or detection steps use light based technologies. In addition, as outlined herein, portions of the internal and/or external surfaces of the device may be coated with a variety of coatings as needed, to facilitate the manipulation or detection technique performed.

Structures within such microfluidic chips—including for example, channels, chambers, and/or wells—generally have dimensions on the order of microns, although in many cases larger dimensions on the order of millimeters, or smaller dimensions on the order of nanometers, are advantageous.

In certain embodiments of the present invention, microfluidic chips are provided including at least one separation channel. The separation channel is configured to facilitate the chemical or physical separation of species in the channel. Separations of interest include, but are not limited to capillary zone electrophoresis, liquid chromatography, affinity chromatography, capillary gel electrophoresis, isotachophoresis, capillary electrochromatography, micellar electrokinetic chromatography, and isoelectric focusing, as known in the art. Accordingly, in some embodiments, it is desirable to have as long of a channel as feasible given the desired size of the microfluidic chip and resultant device. Accordingly, in one embodiment, a spiral channel is provided having a plurality of concentric loops to increase the length of channel per area of the microfluidic chip. Other configurations include curves, arcs, serpentine configurations, and the like.

Similarly, it is desirable for ‘plugs’ or ‘zones’ of, such as a separated component of the sample, to remain distinct as they traverse the separation channel. Accordingly, in some embodiments one or more curves in the separation channel are implemented as low-dispersion curves, described for example in U.S. Pat. No. 6,270,641, PCT Application Number 00/09722, and U.S. patent application Ser. No. 09/707,337, filed 6 Nov. 2000, all of which are hereby incorporated by reference. Briefly, turns, tees and other junctions are provided that produce little dispersion of a sample as it traverses the turn or junction. The reduced dispersion results from contraction and expansion regions that reduce the cross-sectional area over some portion of the turn or junction. Sample dispersion in turns and junctions in then reduced to levels comparable to the effects of diffusion.

In embodiments of the present invention, separation channels are provided being long enough to facilitate the separation of analytes in a fluid. In a preferred embodiment, a 10-cm long separation channel is provided. In other embodiments, the separation channel is between 10 and 30 cm in length, in other embodiments, the separation channel is between 15 and 25 cm in length, and in a preferred embodiment the separation channel is 20 cm in length. Shorter or longer lengths may also be used. The length chosen will vary according to the form factor of the microfluidic chip, sample fluid, electrophoretic media, time desired for separation, and desired resolution of the separation, as known in the art.

Other advantageous channel arrangements and microfluidic chips that may be used with the present invention are described in U.S. application publication No. 2003/0075491 entitled “Compact Microchannel System”, published 24 Apr. 2003, hereby incorporated by reference, and U.S. patent application Ser. No. 09/669,862 entitled “Method and Apparatus for Controlling Cross-Contamination of Microfluidic Channels”, hereby incorporated by reference.

In addition to the separation techniques described above, the microfluidic chip and/or separation channel could be used to perform solid phase extraction, dialysis, sample filtration, analyte labeling, mixing, analyte preconcentration methods, or other sample preparation techniques or other physical or chemical separation techniques, as known in the art. The inlet and outlet ports of the microfluidic device will be placed as needed to perform the desired operation. The separation channel generally serves to separate sample components by the application of an electric field, with the movement of the sample components being due either to their charge or, depending on the surface chemistry of the microchannel, bulk fluid flow as a result of electroosmotic flow (EOF).

As will be appreciated by those in the art, the separation channel generally has associated electrodes to apply an electric field to the channel. Waste fluid outlets and fluid reservoirs are present as required. In some embodiments, electrodes are formed on the chip and are connected to the power supply. In some embodiments, no electrodes are placed on the chip, and the electric field is generated across the channel using electrodes in contact with one or more reservoirs, as described above.

In a preferred embodiment, electrophoretic media is placed in the separation channel. By varying the pore size of the media, employing two or more gel media of different porosity, and/or providing a pore size gradient, separation of sample components can be maximized. Gel media for separation based on size are known, and include, but are not limited to, polyacrylamide and agarose. One preferred electrophoretic separation matrix is described in U.S. Pat. No. 5,135,627, hereby incorporated by reference, that describes the use of “mosaic matrix”, formed by polymerizing a dispersion of microdomains (“dispersoids”) and a polymeric matrix. This allows enhanced separation of target analytes, particularly nucleic acids. Similarly, U.S. Pat. No. 5,569,364, hereby incorporated by reference, describes separation media for electrophoresis comprising submicron to above-micron sized cross-linked gel particles that find use in microfluidic systems. U.S. Pat. No. 5,631,337, hereby incorporated by reference, describes the use of thermoreversible hydrogels comprising polyacrylamide backbones with N-substituents that serve to provide hydrogen bonding groups for improved electrophoretic separation. See also U.S. Pat. Nos. 5,061,336 and 5,071,531, directed to methods of casting gels in capillary tubes.

Further electrophoretic media that may be used in conjunction with embodiments of the present invention may be found in U.S. application Ser. No. 09/310,465, filed 12 May 1999 entitled “Castable 3-dimensional Stationary phase for chromatography” and U.S. application publication No. 2001/0008212 entitled “Castable Three-dimensional Stationary Phase for Electric Field-Driven Applications”, filed 2/28/2001, both of which are hereby incorporated by reference.

One or more microfluidic chips are coupled to a reservoir module, as described above, according to embodiments of the present invention. Accordingly, the microfluidic chip comprises one or more inlets or outlets to allow fluidic communication with one or more reservoirs, and/or one or more ports, as described above. Inlets and outlets are generally structurally similar, and the terms are used interchangeably herein. Each inlet comprises an area of the microfluidic chip in fluidic communication with one or more channels or chambers. Inlets and outlets may be fabricated in a wide variety of ways, depending on the substrate material of the microfluidic chip and the dimensions used. For example, in one embodiment inlets and/or outlets are formed by removing portions of a sealing layer and affixing the sealing layer to a substrate containing chambers and/or channels such that the removed portions of the sealing layer allow fluidic access to one or more channels or chambers.

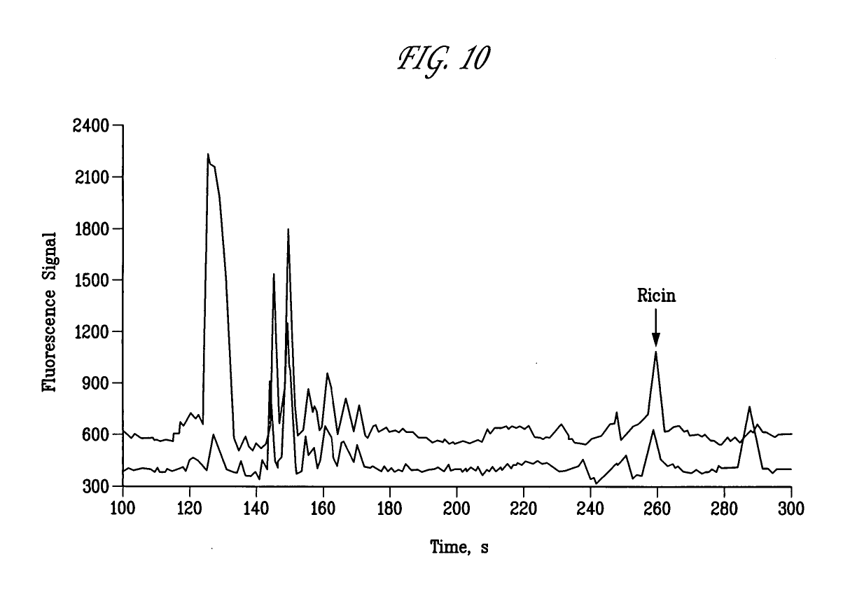

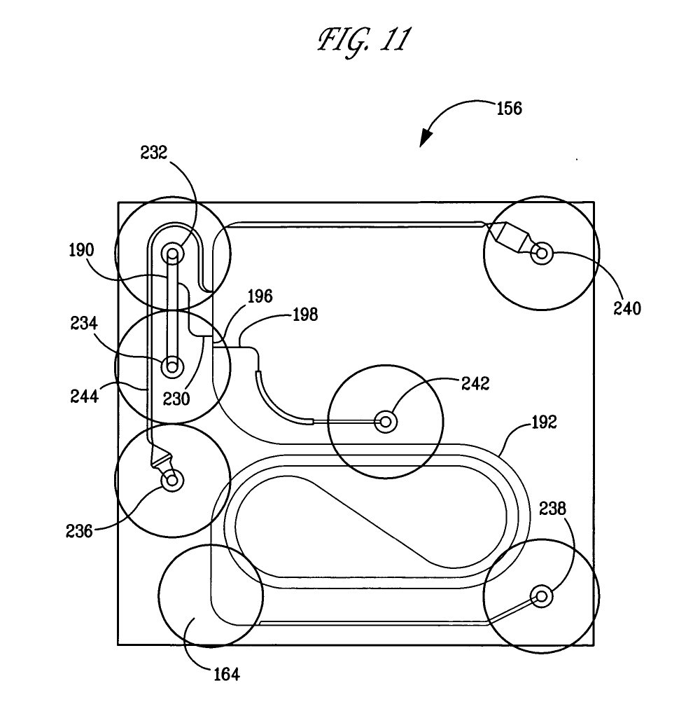

One embodiment of a microfluidic chip according to the present invention is shown in FIG. 11. Various channels are described and referenced, however it is to be understood that the channels are referred to according to their function, and, as is shown, several are contiguous. In general, microfluidic chip 156 contains inlets (or outlets) and channels, and/or chambers. Inlets/outlets allow access to the different reservoirs to which they are connected for the purpose of introducing or removing fluids from the channels/chambers on the microfluidic chip 156. A contiguous fluid path through the inlet allows the passage of electrical current through conductive fluids. It will be understood that the number of inlets/outlets, channels/chambers, their size and configuration, placement, or other design or geometrical arrangement will vary according to the application contemplated on the microfluidic chip. In some embodiments, the configuration of the microfluidic chip will vary according to the physics and chemistry used to perform a microfluidic separation based on a particular analyte characteristic including, but not limited to, electrophoretic mobility, molecular weight, hydrodynamic volume, isoelectric point, or partition coefficient.

Microfluidic chips of the present invention may be fabricated using a variety of techniques, including, but not limited to, hot embossing, such as described in H. Becker, et al., Sensors and Materials, 11, 297, (1999), hereby incorporated by reference, molding of elastomers, such as described in D.C. Duffy, et. al., Anal. Chem., 70, 4974, (1998), hereby incorporated by reference, injection molding, LIGA, soft lithography, silicon fabrication and related thin film processing techniques, as known in the art. In a preferred embodiment, glass etching and diffusion bonding of fused silica substrates are used to prepare microfluidic chips.

A detection module, or ‘detector module’ as used herein, is provided to detect the presence of a target analyte in a portion of the separation channel. In some embodiments, components complementary to those of the detection module are included on the microfluidic chip and/or reservoir. For example, in some embodiments, electrodes for performing electrochemical detection are formed on the interior and exterior surfaces of the microfluidic chip and are in electrical communication with the separation channel and the detector. For example, in some embodiments, additional channels and reservoirs are included in the reservoir module to add reagents to the separation channel for chemiluminescence detection. In some embodiments the method of detecting the presence of target analytes in the separation channel includes, but is not limited to, optical absorbance, refractive index, fluorescence, phosphorescence, chemiluminescence, electrochemiluminescence, electrochemical detection, voltammetry or conductivity. In preferred embodiments, detection occurs using fluorescence and more preferably, laser-induced fluorescence, as is known in the art.

Generally, optical detection of non-fluorescent target analytes involve providing a colored or luminescent dye as a ‘label’ on the target analyte. Fluorescent analytes may be directly detected by optical methods described below. Suitable labels include, but are not limited to, fluorescent lanthanide complexes, including those of Europium and Terbium, fluorescein, fluorescamine, rhodamine, tetramethylrhodamine, eosin, erythrosin, coumarin, methyl-coumarins, pyrene, Malacite green, stilbene, Lucifer Yellow, Cascade Blue™, Texas Red, 1,1′-[1,3-propanediylbis[(dimethylimino-3,1-propanediyl]]bis[4-[(3-methyl-2(3H)-benzoxazolylidene)methyl]]-,tetraioide, which is sold under the name YOYO-1, Cy and Alexa dyes, and others described in the 9th Edition of the Molecular Probes Handbook by Richard P. Haugland, hereby expressly incorporated by reference. Labels may be added to ‘label’ the target analyte prior to introduction into the microfluidic chip, in some embodiments, and in some embodiments the label is added to the target analyte in the microfluidic chip. In general the labels are attached covalently as is known in the art, although non-covalent attachments may also be used.

Further, as is known in the art, photodiodes, confocal microscopes, CCD cameras, or photomultiplier tubes maybe used to image the radiation emitted by fluorescent labels.

In a preferred embodiment, detection occurs using laser-induced fluorescence, as known in the art. Accordingly, in some embodiments, the detector module includes a light source, detector, and other optical components to direct light onto the microfluidic chip and collect fluorescent radiation from the target analyte. The light source preferably includes a laser light source, more preferably a laser diode, and still more preferably a violet or a red laser diode. A violet, or blue, laser diode is preferred in embodiments of the present invention to detect a fluorescamine label on one or more components of the sample. A fluorescamine label is preferred, in embodiments of the present invention, because the fluorescamine label attaches quickly (in milliseconds, in some embodiments) to the components of interest. Accordingly, fluorescamine is preferred in some embodiments to facilitate faster detection of one or more sample components. Violet, or blue, optical sources are accordingly preferred to excite the fluorescamine label. Other color laser diodes may be used, including red laser diodes, as well as other light sources including, but not limited to, laser diodes, light-emitting diodes, VCSELs, VECSELs, and diode-pumped solid state lasers. In some embodiments, a Brewster's angle laser induced fluorescence detector is used. One or more beam steering mirrors are used, in one embodiment, to direct the beam to a detection area on the microfluidic chip. In preferred embodiments, the beam is directed onto the micofluidic chip at Brewster's angle for the material of the chip. For example, in preferred embodiments the microfluidic chip comprises fused silica and the laser diode is directed onto the microfluidic chip at Brewster's angle for fused silica. Beam conditioning optics—including any of, but not limited to lenses, filters, and/or pinholes—may be used to focus the beam onto the microfluidic device. Dye may be injected into the microfluidic chip, in one embodiment, to visualize the location of the beam. A lens is used to collect and collimate the fluorescence and scattered light from the fluidic device. In embodiments where the microfluidic chip comprises a plurality of microchannels, each having a detection area, the detector module comprises a plurality of laser diodes (or other light sources), a plurality of beam steering mirrors to direct light from each diode to a microchannel. The collected light passes through a filter to remove the scattered laser light and the balance of the emissions are detected with a single photomultiplier tube for all channels. In certain embodiments, cross-talk between the detection of each channel can be reduced by alternately pulsing each of the diode lasers so that fluorescence is only generated on one of the fluidic channels at any one time. In a preferred embodiment, the microfluidic chip includes two microchannels, and the detector comprises two laser diodes. However, any number of microchannels and laser diodes may be used, including, for example, 1, 2, 3, 4, 5, 6, 7, 8, 9, or 10 microchannels and a corresponding number of laser diodes.

A power supply is included in embodiments of the present invention to provide the voltages and currents for operating the remaining components—such as pumps and/or valves on the microfluidic chip and the detector module. In some embodiments, the power supply includes a high voltage power supply including a DC-to-DC converter, a voltage-controlled resistor, and a feedback circuit to control the resistor and converter to regulate the voltage of the high voltage supply. By ‘high voltage’ herein is meant voltage sufficient to allow electrokinetic pumping of fluid, as described above. Thus, ‘high voltages’ generally refer to voltages above 100V. Generally, high voltages up to 500 V may be provided, more preferably 800 V, still more preferably 1,000 V, yet more preferably 5,000 V, and still yet more preferably 10,000 V. Embodiments of a power supply suitable for use in the present invention are described in U.S. application Ser. No. 10/414,979 entitled “Modular High Voltage Power Supply for Chemical Analysis”, filed 16 Apr. 2003 and U.S. patent application Ser. No. ______ entitled “Scalable Power Supply”, Docket No. SD-8409, filed Jun. 3, 2003, both of which are hereby incorporated by reference.

In embodiments of the present invention, the power supply is coupled to an external power supply. In other embodiments, the power supply is powered using a portable power supply, such as batteries, solar power, wind power, nuclear power, and the like. Accordingly, in certain embodiments of the present invention, the power supply operates using less than 3V of DC power. In other embodiments, 6 V of DC power are used to power a portable system.

The power supply is coupled to, inter alia, the electrodes in one or more reservoirs. In some embodiments, the power supply is coupled to electrodes located on or in the microfluidic chip. In one embodiment, The power supply is coupled to the electrodes in contact with the reservoirs by way of an electrode plate containing interconnects in electronic communication with the electrodes in the reservoirs and a wire or other electrical connection to the power supply. In this manner, the power supply itself can be disconnected from the reservoir module and/or microfluidic chip and be changed, such that a different voltage may be provided or the module simply replaced. In some embodiments, the power supply is operated in constant current mode wherein the power supply provides a constant output of current by varying the voltage applied by the power supply to the fluid, and therefore the microfluidic channel. In some embodiments, the power supply is operated in constant voltage mode wherein the power supply provides a constant voltage potential to the microfluidic channel and allows the current to vary according to the conductivity of the microfluidic channel. Constant current mode generally maintains a constant electric field between the electrodes and is generally preferred, as migration times, without being bound by theory, are generally more repeatable and reliable.

In further embodiments of the present invention, one or more data processors are provided in communication with the detection module and power supply to collect and/or analyze data generated by the system. In some embodiments of the present invention, a user interface is coupled to a dataprocessor. The user interface may include a visual display, for example, in one embodiment an LCD display, a keypad, one or more buttons, a mouse, and/or the like. In some embodiments, the user interface is menu-driven. The user interface allows a user, in some embodiments, to view data, to select the detection technique, to determine which separation channel to use, to determine which of a plurality of detection modules to activate, and the like.

In preferred embodiments of the present invention, at least one reservoir module, a microfluidic chip, a power supply, and a detector module are interconnected as generally described above and packaged within a single housing. In some embodiments, a plurality of microfluidic chips are provided within a single housing along with a plurality of power supplies and a plurality of reservoir modules and detector modules. The individual modules can be replaced without removing or exchanging the remaining modules. Dovetail rails and other mechanical assemblies facilitate the swapping of modules in and out, in some embodiments. In some embodiments the housing containing the modules further comprises heat sinking and/or ventilation, as known in the art, to maintain the various modules at or near ambient temperature. In some cases, heating and/or cooling elements may also be provided. The housing containing the modules is desirably rugged and portable, in preferred embodiments.

Methods for detecting a target analyte in a sample according to embodiments of the present invention generally proceed as follows. A sample is brought into contact with a sample introduction port. In some embodiments, the sample is injected through the housing in which the reservoir module is placed. That is, a sample introduction port is provided in the housing in fluid communication with the sample introduction port in the reservoir module. In some embodiments, no external housing is present. A sample is injected into the sample introduction port of a reservoir module filling the injection inlet, sample loop channel and sample inlet of the microfluidic chip. Excess sample moves through the sample inlet, through a needle and into a sample waste reservoir.

One or more channels in the microfluidic device may be flushed. A syringe containing separation media is connected to a channel flush port in the reservoir module. Separation medium is pushed into the reservoir module entering the microfluidic chip at the flush inlet, filling all channels/chambers and exiting the chip inlets. Optionally, separation medium is also injected into the sample loop channel with a syringe through the introduction port in some embodiments.

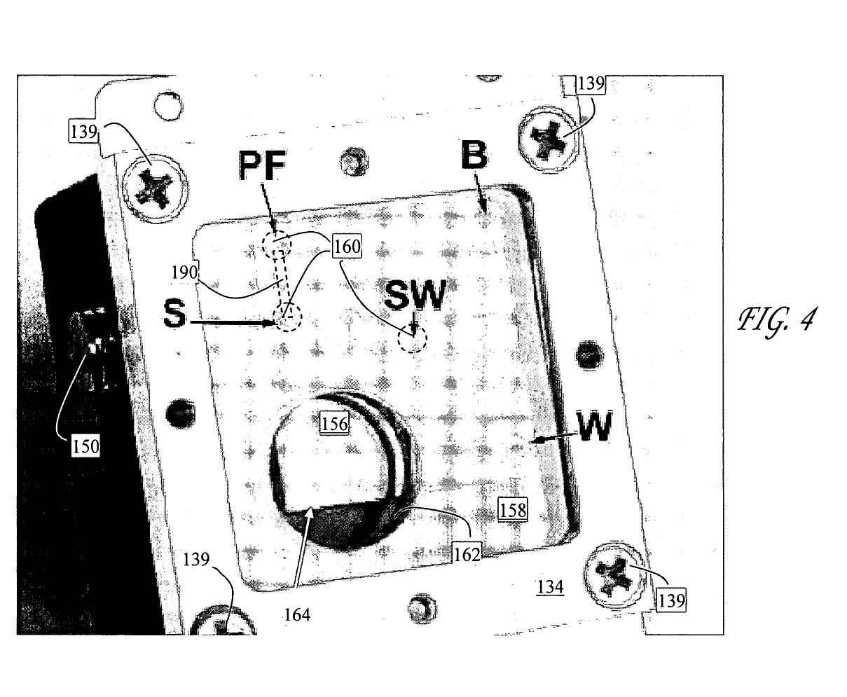

A microfluidic separation is performed. The particular procedure for performing a microfluidic separation will vary according to the type of separation performed and the microfluidic chip configuration. In one embodiment, the separation proceeds as follows, with reference to FIG. 4, a voltage and current are applied to the sample electrode S and sample waste electrode SW positioned in the sample reservoir and sample waste reservoir, respectively. Referring also to FIG. 11, sample in the sample loop channel moves under the influence of the electric field and fills the sample channel, injection cross and begins to fill the sample waste channel. In one embodiment, a smaller voltage and current is applied to the buffer electrode and waste electrode, positioned in the buffer reservoir and waste reservoir, respectively, to help confine sample in the injection cross; a pinched injection as is known by those familiar with the art. The voltages and currents causing the electrokinetic injection are turned off. A voltage and current are applied to the buffer electrode and waste electrode, positioned in the buffer reservoir and waste reservoir, respectively, in one embodiment. The sample contained in the injection cross moves into the separation channel and begins to divide into individual analyte zones. In one embodiment, a smaller voltage and current are applied to the sample electrode and sample waste electrode to prevent sample from spilling from the sample channel and sample waste channel into the injection cross and the separation channel; anti-siphoning voltage as is known by those familiar with the art. The separation voltage is applied until the individual analyte zones pass through the separation channel, past the detection area and into the waste channel. The time between the application of the separation voltage and the appearance of the center of the analyte zone in the detector signal defines the time for the analyte in the sample, in one embodiment, and is indicative of the presence of the analyte in the sample. Time may be converted into a characteristic for the component, such as electrophoretic mobility, molecular weight, hydrodynamic volume, isoelectric point, or partition coefficient, in some embodiments to facilitate determination of the component and/or analyte. The analysis process may be repeated by injection of a second sample into the sample loop channel, in some embodiments.

Accordingly, an elution spectrum is generated according to the particular separation technique used. The elution spectrum generally contains a plurality of peaks, each indicating a migration time of one or more sample components. The migration time is indicative of a separation characteristic, as determined by the separation technique used. Separation characteristics include, for example, the component characteristics described above. One or more calibrations may also be performed, as generally known in the art. A preferred calibration is described further below.

At least one component of the separated sample is detected. Generally, a ‘component’ of the sample may be any of the target analytes described above. Some target analytes, however, will include several separated ‘components’, such as viruses (which, for example, include a plurality of proteins). The component may be directly detected—for example, by tagging the component with a fluorescent label. In some embodiments, a substance indicative of the presence of a component or target analyte may be detected—for example, using an eTag™ reporter (ACLARA Biosciences™; Mountain View, Calif.). Based on the detected component, or in some embodiments, based on the detection of a plurality of components, the target analyte is identified. The identification generally proceeds by correlating the signal generated by the detection module with a signal for a known target analyte, or of components of interest. In some embodiments, if a correlation cannot be made between the signal generated by the detection module and a signal for known target analytes, the presence of an analyte is reported, but its identity remains unknown.

Further in some embodiments, the quantity of target analyte in the sample is also reported. The quantity of analyte is determined by comparing the signal generated by the detection module with a calibration curve for the analyte of interest.

The presence of the target is then indicated on an output interface of the portable device. The indication may include, but is not limited to, a visual display, an audible sound, a tactile signal, or any combination thereof.

In embodiments where a plurality of microchannels are provided on a microfluidic chip, a second portion of the sample fluid may be transported to a second separation channel, and a detection area on the second separation channel is interrogated with the detection module. In some embodiments, a plurality of microfluidic chips are provided, each with one or more separation channels. A single sample may be injected and multiplexed onto each chip, in one embodiment. In another embodiment, separate samples, or portions of a single sample, are injected, one into each microfluidic chip. In some embodiments where a plurality of microfluidic chips are provided, one or more microfluidic chips are configured to perform the same or different microfluidic separation method and, where one or more samples are introduced into the device, the distribution of samples among microfluidic separation methods can be in any association.

The detector module, power supply, separation module and/or microfluidic chip can be removed from the system and replaced, or a second module inserted. Changing detector modules, for example, allows for a change in light source, wavelength, or light intensity from one separation measurement to the next or changing from one detection method to another. Change microfluidic chips allows for a change in the application of the system. Changing power sources provides a change in voltage and/or power level.

Suitable microfluidic chips further include an electrokinetic pump for transporting biological molecules to the microchannel separator. Suitable electrokinetic pumps are provided by transporting a liquid sample into a suitable fluidic holding channel that is in fluid communication with a suitable electrokinetic injection channel. The electrokinetic injection channel is also in fluid communication with a suitable microchannel separator that contains a suitable offset T sample loop for loading a sample plug in the microchannel separator. The T sample loop also contains a waste channel for removing excess sample liquid. Sample fluid is typically pressure injected into the holding channel and buffer solution is typically provided into the electrokinetic injection channel. The electrokinetic pump is actuated by applying a high electric potential between the fluidic holding channel and the sample waste channel to provide a liquid sample in the offset T sample loop. The transport of fluids on microfluidic chips using electrokinetics is further described in International Application No. PCT/US00/30422, “Microfluidic Devices with Thick-Film Electrochemical Detection”, filed Nov. 3, 2000, the entirety of which is incorporated by reference herein.

Suitable electrophoretic microchannel separators used in certain embodiments of the present invention are capable of separating proteins using a electric field. Suitable electric fields are typically operated in a controlled mode, such as a constant voltage mode. Preferably, the suitable electric field on the microchannel separators are operated in a constant-current mode. Suitable electrophoretic microchannel separators typically use a high voltage.

Suitable electrophoretic microchannel separators are typically provided with a suitable separation medium. Examples of suitable separation media generally include polymeric materials, ceramic materials, or both. Suitable separation media can also have a variety of forms, such as gels, particulates, or both. Suitable separation media are capable of being fluidically conducted into separation microchannels, such as by pressure injection or electrokinetic pumping. Typically, a polymeric separation media, such as aqueous polyethylene oxide, is used as a microchannel separation media. Various suitable polymeric separation media are commercially available.

Suitable detectors used in the present invention typically giving rise to signals that are correlatable to the concentration and separation time of the separated proteins. Suitable detectors include a light source to provide a light beam for interrogating the molecules in the sample liquid, such as a laser. Suitable detectors also typically include optics for collimating and directing the light beam to the sample, and an observation lens for collecting optical signals received from the interrogated molecules. Particularly preferred detectors are provided in U.S. patent application Ser. No. 10/633,794, “Optical Detector System”, filed Aug. 4, 2003, the entirety of which is incorporated by reference herein.

A suitable photomultiplier tube (“PMT”) or a charge-coupled device (“CCD”) is also typically included in a suitable detector for detecting the optical signals and converting them to electrical signals that can processed by a data processor. A suitable PMT includes any PMT that is capable of detecting separation peaks with widely varying amplitudes. The ratio of the largest detectible peak height divided by the smallest detectible peak height is the system dynamic range. Suitable packaged PMTs typically have a system dynamic range of at least about 1000 to about 4000 counts. Suitable PMTs are may be of the fixed-gain type, although dynamic-gain PMTs are preferred. PMTs having too low a system dynamic range typically results in the required gain to detect small peaks causing saturation and clipping of large peaks. Preferred PMTs having increased optical detection dynamic range are provided by software-driven PMTs that implement automatic gain control (AGC). AGC PMTs are typically preferred in embodiments of the system having significant background light. Suitable AGC PMTs are capable of having dynamic ranges that are relatively larger than the dynamic range of a suitable analog-digital converter. In one possible scheme, suitable AGC PMTs are provided with an initial fixed PMT gain to initially achieve a reasonable background level. Suitable AGC software typically first computes the required PMT voltage to yield an optical gain equal to about half the initial value. During operation in which an optical signal is received by a detector emanating from the detection region of a suitable microfluidic chip by after sample separation, the AGC software typically is capable of constantly comparing the PMT output to a threshold value. The threshold value is typically at least about 50 percent of the total A/D counts, more typically at least about 75 percent of the total A/D counts, and even more typically at least about 85 percent of the total A/D counts. By “A/D counts” is meant that number of digital values into which the input range is equally divided. Suitable analog-digital converters typically have at least about 1000 total A/D counts, more typically at least about 2000 total A/D counts, and even more typically at least about 4000 A/D counts. Typically when the output signal exceeds the threshold value, the AGC software lowers the PMT gain to a value lower than the current gain, typically to a value less than about 80 percent, more typically to a value less than abut 60 percent, and even more typically to a value of exactly 50 percent of the current gain (“the PMT gain reduction upon threshold value”), and multiplies the resulting digital value accordingly. Preferably when the output signal exceeds the threshold value, the AGC software lowers the PMT gain to a value of about 50 percent of the current gain, and multiplies the resulting digital value by two. In a preferred embodiment using a 4096-count converter, AGC software is provided with a rising threshold value of 3500 counts and a PMT gain reduction upon threshold value of 50 percent. Suitable AGC PMTs typically have measurement and gain adjusting cycles on the order of about 10 to 100 milliseconds.

In a preferred embodiment, there is provided an AGC PMT having a dynamic range of 4096 counts, an increasing value threshold of 3500 counts, and a PMT gain reduction upon threshold value of 50 percent. During operation, for example, this AGC PMT is capable of receiving an optical signal equivalent to 3600 counts and the AGC software in response reduces the PMT gain by 50 percent to achieve an optical signal equivalent to 1800 counts. The resulting signal (count) will appear as 3600 counts when multiplied by two, resulting in a seamless transition between the gains. Reduction of the optical gain accordingly avoids saturation in preferred PMTs. Typical maximum PMT output is about 4.096 V (4096 A/D counts resulting from a 12-bit A/D converter at 0.001 V per count). Suitable PMTs typically have a larger dynamic range than the A/D converter. The signal bit value of suitable PMTs is typically at least two bits greater than, even more typically at least three bits greater than, and even further typically at least four bits greater than the signal bit length of the A/D converter. In preferred embodiments the PMT has a signal bit length of 16 in the final converted digital value and the A/D converter has a signal bit length of 12. With the PMT gain halved in the preferred embodiment, the AGC software is capable of comparing its output to 1700 counts and when the value drops below this number, the AGC software is capable of restoring the PMT to its initial value and accordingly adjusts the multiplication of the final digital value. A several hundred count hysteresis is typically provided to prevent oscillation of the digital value that is capable of arising from noise.

In a preferred embodiment, a custom PMT power supply having a fast response to changes in the high voltage program value is provided. Standard PMT power supplies typically respond to an increase in the high voltage in approximately 100 milliseconds, but require 3-4 seconds to respond to a decrease. In a preferred embodiment, the digitization sample rate of the PMT is typically at least about 1 Hz, and preferably at least about 10 Hz. Such preferred PMTs typically require a PMT power supply that can change value and stabilize in less than about 50 milliseconds. Suitable high voltage supplies typically are capable of providing both source current and sink current into the PMT dynode network. Preferred PMT and power supplies can change optical gain in no more than about 20 milliseconds. The effective result of this rapid scaling is the doubling of the dynamic range of the detector, so that rather than a maximal 4096 counts the detector scales to 8200 counts.

Suitable data processors are provided for correlating the signals received from a suitable PMT or CCD device to molecular signatures of known biological samples. In certain embodiments, it is preferred that the molecular signatures include the protein signatures of known biological samples, however any biologically-discriminating molecular signature can be used, such as nucleic acids. Preferred biological samples include molecular portions of viruses, such as viral proteins. Suitable data processors will include data registers for storing calibration data, such as separation time and suitable molecular parameters, such as molecular weight. Suitable data processors typically include data registers for storing processing algorithms for generating calibration data from calibration test results. Suitable data processors are typically capable of converting separation information signals, such as separation time and detection intensity to molecular parameter information of biological molecular samples, such as molecular weight. Typically, the systems include a housing, and the data processor is contained within the housing of the system. In alternate embodiments, the data processor can be provided external to the housing. In such embodiments having external data processors, the necessary information signals can be transported from the system to the data processor by a suitable information transmission means, such as by electrical wire, optical wire, or radio wave. Typical embodiments of the system of the present invention further include an information display coupled to the data processor.

Electrical power can be provided to the systems of the present invention using externally supplied power sources (examples being an AC wall outlet, battery pack, fuel cell, solar panel, or other type of electrical generator), or by using a self-contained power source (examples being batteries or a fuel cell). A minor portion of the electrical power is typically used to operate a data processor and data display. A major portion of the electrical power typically provides the high voltages necessary to transport fluids on the microfluidic chip. Examples of type of transport of fluids on microfluid chips include, inter alia, transport of sample liquids, transport of buffer liquids, transport of separation media, transport of separation media precursors, and separation of sample liquids. Multi-channel voltage sources are typically provided and controlled to transport, separate, or transport and separate, fluids through in each of the microchannels on the microfluidic chips.

In certain preferred embodiments of the present invention, the system includes at least one power supply that is capable of generating at least one full-scale stepped voltage in at most 20 milliseconds and capable of measuring at least one current in at most 20 milliseconds. Such power supplies are preferably used for controlling separation of sample analytes in a constant current mode. Preferred systems according to the present invention include power supplies that further include an embedded microprocessor capable of measuring many electric currents at least once every 100 milliseconds and capable of updating at least one voltage at least once every 100 milliseconds. Preferably, such systems include embedded microprocessors that are capable of measuring many electric currents at least once every 50 milliseconds and is capable of updating at least one voltage at least once every 50 milliseconds. Multi-channel power supplies are particularly useful, for example, those that include embedded microprocessors that are capable of measuring at least ten electric currents at least once every 100 milliseconds and are capable of individually updating at least ten voltages at least once every 100 milliseconds. Certain preferred power supplies used in the systems of the present invention include embedded microprocessors that include a current control feedback algorithm and a timer interrupt. In the preferred power supplies, the feedback algorithm typically is capable of operating on updated voltages and current measurements by operation of a digital-to-analog converter coupled to the timer interrupt.

Preferred power supplies for applying electrical separation voltages and currents to the separation channel typically include a control mechanism to accomplish constant current control during analyte separation. Although suitable control mechanisms can be accomplished using hardware, the control mechanism is preferably provided using software. Software control of the electrical separation voltages and currents offer greater flexibility and simpler design for improving miniaturization, power savings and reliability of the systems and methods of the present invention. As described further in the examples provide herein, it is preferred to control the current on the separation column (separation channel). In preferred microfluid chips, the separation channel is typically the longest fluidic channel, which requires the application of voltages typically at least about 2,000 V, more typically at least about 3,000 V, even more typically at least about 4,000 V, and even further typically at least about 5,000 V to perform separations in a reasonable time. The applied voltages are typically less than about 15,000 V and even more typically less than about 10,000 V. The applied separation column voltage is preferably in the range of from about 5,000 V to about 10,000V. The voltage signals applied to the channels may be multiplexed, although multiplexing is typically not required in the preferred power supplies.

In another embodiment of the present invention there is provided a system that includes an injection port, a microfluidic chip, a detector and a data processor. In this embodiment, the injection port is provided for receiving biological samples that include biological macromolecules. Typical injection ports are capable of receiving biological samples through a tubular pressurized port, such as a syringe. Suitable microfluidic chips are typically provided in fluid communication with the injection port, such as by way of tubing or channels placed between the injection port and a suitable inlet port on the microfluidic chip. Suitable microfluidic chips also optionally include a preconcentrator in fluid communication with the injection port. Suitable optional preconcentrators are described herein

Suitable electrophoretic microchannel separators are capable of separating biological macromolecules according to their molecular weight, mass to charge ratio, charge, or any combination thereof. In certain preferred embodiments, the electrophoretic microchannel separators are capable of being operated using a controlled electric field. Preferably, such controlled electric fields are capable of being operated in a constant-current mode. Suitable detectors capable of detecting the presence of said separated biological macromolecules are also provided in these embodiments. Typically, the detectors give rise to signals being correlatable to the concentration and separation time of the separated biological macromolecules as described herein. The systems of the present invention also typically include one or more data processors for correlating the detector signals to a biological macromolecular signature of a biological entity. In certain preferred embodiments that include a preconcentrator, typical preconcentrators may include a porous surface in fluid communication between a first channel provided in the microfluidic chip and a second channel provided in the microfluidic chip. Preferably, the porous surface includes a cover material bonded to a rough surface. Preconcentrators are further described herein.

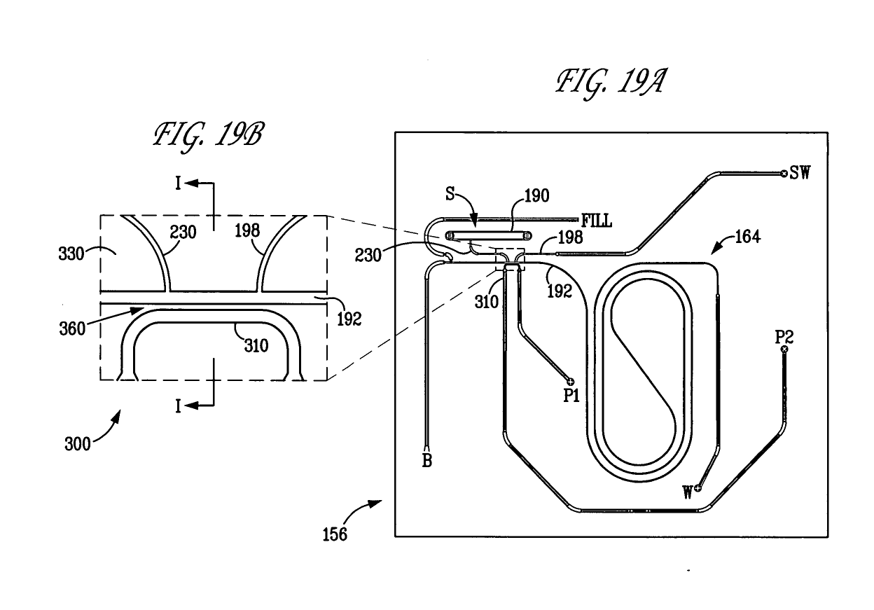

Suitable microfluidic chips typically include an injection port for receiving liquid samples containing analytes, such as aqueous solutions of biological molecules. Referring to FIGS. 19A and 19B, suitable microfluidic chips (156) may include a preconcentrator (300) for increasing the concentration of the biological molecules in the liquid samples. Preconcentrators are typically provided at the upstream end of a microchannel separator (192), although the preconcentrator may be provided downstream from the microchannel separator. The preconcentrator is also typically provided in a location along the microfluidic path that is upstream from a suitable detection region (164). In certain embodiments of the systems of the present invention, preconcentrators are provided at the upstream end of the microchannel separator.

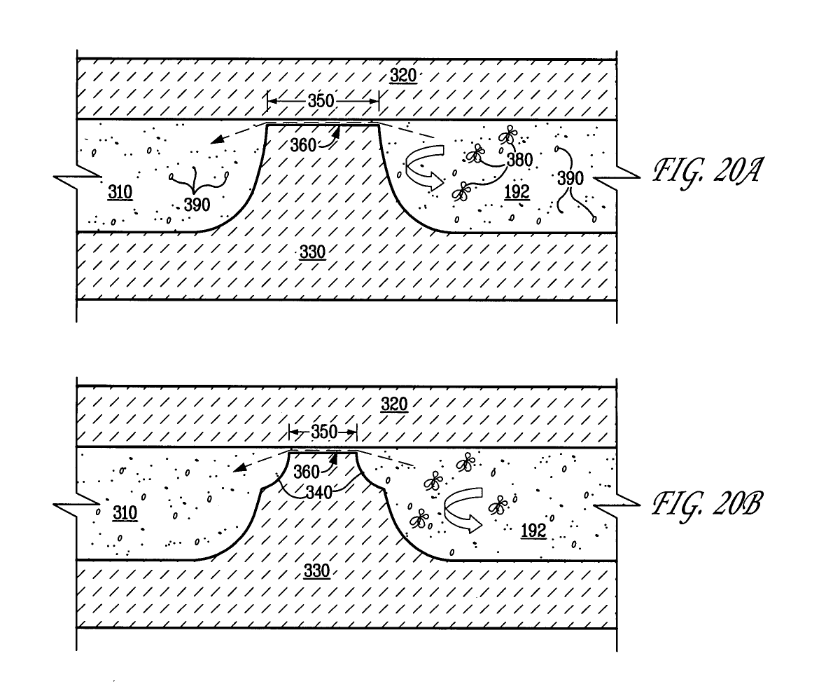

FIGS. 20A and 20B provide a view along direction I-I of FIG. 19B. Referring to FIGS. 20A and 20B, the preconcentrator (300) typically includes a porous surface (360) in fluid communication between two channels (192, 310) provided in the substrate of the microfluidic chip (330), such as between the microfluidic separation channel (192) including analyte molecules and ions, and a second channel (310) containing a buffer solution or a suitable solvent, such as water. Various types of porous surfaces (360) can be provided between two channels for providing a preconcentrator. Referring to FIG. 20(A), a porous surface is provided by a cover material (320) being bonded to the top surface of the microfluidic chip, the porous surface being made of the surface of the substrate of the microfluidic chip (330) and the porous surface (360) being in fluid communications with the two channels (192, 310). A suitable root-mean-squared (“RMS”) surface roughness of the top surface of the microfluidic chip prior to bonding is typically about 100 Angstroms (Å). The resulting porosity of the porous surface is typically of a suitable size so that large analyte molecules (380) (not drawn to scale), such as proteomic substances or nucleic acids, are retained in the microfluidic separation channel (192) and ions in the buffer solution (390) (not drawn to scale) are able to pass from the separation channel (192), through the porous surface (360), and into the second channel (310). Such preconcentrators are particularly desirable for concentrating dilute viral protein samples. The porous surface is preferably provided by applying a cover glass (320) to a microfluidic chip having a narrow gap (350) between the two channels etched in the substrate of the microfluidic chip (330). The narrow gap width (350) is measured as the distance along the cover material between the two channels. The two channels (192, 310) are typically provided by standard substrate etching methodologies known in the microfluidic arts. Preferably, a preconcentrator is incorporated in the microfluidic chip design. The narrow gap between the two channels is typically less than about 20 microns, even more typically less than about 15 microns, and even more typically less than about 12 microns. While gaps smaller than about 10 microns can be used, gaps that are too small may lead to incomplete bonding of the microfluidic chip to the cover material. In this regard, the narrow gap is typically a least about five microns, more typically at least about seven microns, and even more typically at least about 9 microns wide. Without being bound by a particular theory of operation, the flow of ions through the porous surface under the influence of an electric field across the narrow gap (not shown) gives rise to congregation of the large analyte molecules in the portion of the separation channel (192) that is proximate to the porous surface (360). Over time, this congregation gives rise to an increase in concentration of analyte molecules in the preconcentrator (300). After the analyte molecules have been sufficiently concentrated, the electric field across the narrow gap is typically reduced to release the analyte molecules into the separation channel (192).

Referring to FIG. 20(B), in certain preferred embodiments, each of the two channels (192, 310) further include a shallow etched region (340) in contact with the narrow gap (350). In this embodiment, a two-level etch can be provided that allows improved control over the effective gap width (350). Two-level etched gaps typically provide smoother edges compared to a single-level etch of the two deep channels. Smoother edges typically result from shorter etch times required for the second etch in channels having a two-level etch. A suitably bonded narrow gap is typically tested by observation of hydrostatic pressure filling of the separation channel with separation media. A suitably narrow gap is typically observed if separation media does not seep into the second channel (310) during filling of the separation channel (192).

Suitable preconcentrators are provided using a bonding process that bonds a cover material to a substrate. Suitable cover materials are clear and flat and are composed of materials that are suitable for preparing the microfluidic chip substrates as described herein. In certain embodiments the cover material can be composed of the same material as that of the substrate. Typically, the surfaces of the cover material and the substrate are flat and are prepared by contacting the surfaces with a suitable base, such as NaOH 40 wt. % for 15 minutes. Opposing surfaces are then brought together to effect hydrostatic bonding. The hydrostatically-bonded cover and substrate are then thermally bonded. Suitable bonding processes typically include raising the temperature of the hydrostatically-bonded cover and substrate to the softening point. For substrates and covers prepared from different materials, the desired temperature will be that of the material having the lower softening point. For substrates and covers prepared from different materials, the desired temperature will be that of the material having the lower softening point. Typically, the substrates and covers are prepared from materials having similar softening points, which is typically provided using the same materials for the cover and substrate. For example, substrates and covers prepared from fused silica typically are thermally bonded at about 1150° C. for about five hours at ambient pressures in a nitrogen environment. Covers and substrates prepared from Borofloat™ glass typically are thermally bonded at about 610° C. for about five hours at ambient pressures in a nitrogen environment.

The preconcentrators can be used to achieve concentration factors typically at least about a factor of two, more typically at least about a factor of five, even more typically at least about a factor of ten, further typically at least about a factor of 20, and even as high as a factor of 100 in approximately one minute. Longer preconcentration times typically lead to greater concentration factors. Preconcentrating times are typically less than about 10 minutes, more typically less than about five minutes, even more typically less than about three minutes, and further typically less than about two minutes. Typically the preconcentrating times are at least about 10 seconds. The preconcentrators of the present invention are suitable for use with microchannel separators operating with capillary gel electrophoresis (“CGE”) or capillary zone electrophoresis (“CZE”). When operating with CZE, electroosmotic flow (“EOF”) is typically reduced to about zero across the narrow gap during separation. An EOF of about zero helps to prevent flow of fluid through the narrow gap, which aids in preventing pressure generation in the microfluidic chip.

In a particularly preferred embodiment of the present invention, there are provided systems that include at least one microfluidic injection port, at least one microfluidic chip, at least one laser-induced fluorescence detector, and at least one data processor. In this embodiment, the microfluidic sample injection port is typically capable of receiving a liquid including proteomic substances, such as viral proteins in an aqueous medium. The microfluidic chip preferably includes a preconcentrator in fluidic communication with the injection port. Suitable preconcentrators of this embodiment typically include a porous surface in fluid communication between a first channel provided in the microfluidic chip and a second channel provided in the microfluidic chip. The first and second channels typically comprise deep etched portions in the microfluidic chip and shallow etched portions in the deep etch portions. In this embodiment, the shallow etched portions are arranged adjacent to the porous surface. The porous surface and the first and second channels of the microfluidic chip typically include a cover material bonded to a rough surface on the microfluidic chip, the rough surface being contiguous to the shallow etched portions. In this embodiment, a microchannel capillary zone electrophoresis separator or a microchannel capillary gel electrophoresis separator is typically provided in fluid communication with the preconcentrator.

In this embodiment, at least one laser-induced fluorescence (“LIF”) detector capable of detecting the presence of said separated biological macromolecules is also provided. Typically, the laser-induced fluorescence detectors give rise to signals being correlatable to the concentration and separation time of separated biological macromolecules, which are preferably proteomic substances, as described herein. Such detectors typically include relatively short wavelength laser light sources and detect relatively longer wavelength fluorescence signals from the liquid sample. The systems of the present invention also typically include one or more data processors for correlating the detector signals to a biological macromolecular signature of a biological entity.

In another embodiment of the present invention, there is provided a system including at least one separation module and a system of power supplies. In this embodiment, suitable separation modules include a microfluidic sample injection port, a fluidic system and a microfluidic fluorescence detector. Suitable microfluidic sample injection ports are typically capable of receiving a liquid sample under pressure, such as by insertion of a syringe needle tip into a cylindrical pressure fitting. Any of a variety of pressure fittings known in the fluids handling art can be suitably adapted for use in this aspect of the present invention. Suitable microfluidic sample injection ports are typically capable of handling liquid samples that include proteomic substances. Suitable fluidic systems of the separation module are capable of electrokinetically transporting the sample liquid. Electrokinetic transportation of sample liquids is described further herein. Electrokinetic or electrophoretic transportation typically involves the transport of sample liquids or negative or positive charged ionic analytes under the influence of an electrical potential or field through any one or more of channels, ports, reactions zones, mixing zones, separation zones, holding zones, or any combination thereof, that are located on or among one or more microfluidic chips. Typical electrokinetic transportation in this embodiment suitably transports liquid samples from a holding channel located on a microfluidic chip towards one or more separation channels located on the chip. Suitable fluidic systems of the at least one separation modules of this embodiment further are typically capable of separating proteomic substances by molecular size. Such separating is typically accomplished by providing a separation media in a separation microchannel on a microfluidic chip, and transporting the liquid sample through the separation media to effect separation of the liquid sample components. Suitable separation media includes, for example, any commercially available protein separation gel, such as polyethylene oxide protein separation gel. Typically, the separation channels of the fluidic system are capable of separating proteomic substances using a suitable electrophoresis methodology, such as capillary gel electrophoresis, capillary zone electrophoresis, or both.

In certain preferred embodiments, the operation of electrokinetic and electrophoretic methodologies typically includes application of high voltages that are on the order of about 100 to 1000 volts/cm. Such voltages may be suitably provided by one or more power supplies. Preferably, the system includes at least one power supply that is capable of generating at least one full-scale stepped voltage in at most 20 milliseconds and capable of measuring at least one current in at most 20 milliseconds. Preferred systems according to the present invention include power supplies that further include an embedded microprocessor capable of measuring many electric current at least once every 100 milliseconds and capable of updating at least one voltage at least once every 100 milliseconds. Preferably, such systems include embedded microprocessors that are capable of measuring many electric current at least once every 50 milliseconds and is capable of updating at least one voltage at least once every 50 milliseconds. Multi-channel power supplies are particularly useful, for example, those that include embedded microprocessors that are capable of measuring at least ten electric currents at least once every 100 milliseconds and are capable of individually updating at least ten voltages at least once every 100 milliseconds. Certain preferred power supplies used in the systems of the present invention include embedded microprocessors that include a current control feedback algorithm and a timer interrupt. In these power supplies, the feedback algorithm typically is capable of operating on updated voltages and current measurements by operation of a digital-to-analog converter coupled to the timer interrupt.

The separation modules of certain preferred embodiments further include one or more microfluidic fluorescence detector capable of detecting the concentration and separation time of biological substances, such as proteomic substance, in a liquid sample. Suitable detectors include a laser-induced fluorescence (“LIF”) detector capable of detecting the presence of proteomic substances, although any type of fluorescence detector should be suitable. Typically, the laser-induced fluorescence detectors give rise to signals being correlatable to the concentration and separation time of separated proteomic substances. Such detectors typically include relatively short wavelength laser light sources and detect relatively longer wavelength fluorescence signals from biological molecules, such as proteomic substances, in the liquid sample. Biological molecules in the liquid samples are typically labeled prior to separation with a suitable fluorescent dye, such as fluorescamine dye. The systems of the present invention also typically include one or more data processors for correlating the detector signals to a biological macromolecular signature of a biological entity.

In certain preferred embodiments the system includes a housing that contains the separation module, the power supply and a power source. Suitable power sources that may be located within the housing typically include batteries and fuel cells, or both. Such systems of the present invention that contain the power source within the housing are typically portable and are capable of hand-held operation. Alternatively, the power source may be located external to the housing, such as by using a portable power unit connected to the system by a power cable, or by using an electrical wall outlet. Suitable portable power units include battery packs, fuel cells, fossil-fuel powered electrical generators, ethanol-fuel powered electrical generators, hydrogen-powered generators and fuel cells, bioenergy generators, nuclear-fuel powered electrical generators, and solar cells.

The present invention also provides processes for detecting the presence or absence of biological agents in a sample. The processes of the present invention are capable of detecting a variety of biological agents, including viruses, bacteria, prions, natural toxins derived from biological sources, such as ricin, as well as synthetic toxins. These processes typically involve solubilizing one or more components of a biological agent in a sample, labeling at least a portion of the components for detection, transporting the solubilized components to a microfluidic chip, separating and detecting the solubilized components using a suitable microchannel separator on the microfluidic chip, and analyzing the results to identify the presence or absence of a particular biological agent in a sample.

In one embodiment of the present invention there is provided a process for identifying a virus that includes solubilizing at least a portion or the proteins of a virus, labeling the proteins, and separating and analyzing the labeled proteins using at least one microchannel electrophoretic separator on a microfluidic chip. Viral proteins are suitably solubilized using any of the commercially available lysing solutions that are suitable for proteins. Preferably the viral coat proteins are solubilized. The lysed proteins solutions are typically prepared with a lysis buffer and a small amount of surfactant, such as sodium lauryl sulfate, is added to improve wetting of the aqueous sample solution to a suitable microfluidic chip that is described further herein. At least a portion of the solubilized proteins are labeled to provide labeled proteins on the microfluidic chip. Labeling can be carried out using any one of a variety of commercially-available labeling solutions or kits, and suitably include the addition of protein-reactive dye. The labeling step typically includes chemically reacting the proteins with a labeling reagent, which is typically an amine-derivatized reagent, such as fluorescamine dye.

In certain embodiments of the present invention, at least a portion of the labeled proteins are subsequently electrokinetically injected into at least one microchannel electrophoretic separator on the microfluidic chip. Electrokinetic injection is typically conducted by providing a liquid sample in a holding channel on a microfluidic chip and applying an electrical potential between the holding channel and a reservoir in fluidic communication with the microfluidic electrophoretic separator to electrokinetically direct a portion of the liquid sample from the holding channel into the separator. Further details concerning electrokinetic injection can be found in U.S. patent application Ser. No. 2004/0028567A1, “High throughput screening assay systems in microscalefluidic devices”, the entirety of which is incorporated by reference herein.

Certain embodiments of processes of the present invention also include electrophoretically separating at least a portion of the labeled proteins. Electrophoretically separating a portion of the labeled proteins typically involves using at least one electrophoresis separation methodology that is suitable within a microfluidic channel. In certain embodiments, the electrophoretically separating step may include using at least two electrophoretic separations in parallel or in series. In embodiments having at least two electrophoretic separations, the separations may be conducted on the same or different microfluidic chips. When two or more electrophoretic separations are provided on two different microfluidic chips, the chips may be mounted in the same or different separation modules as provided elsewhere herein. Preferably, the at least two parallel electrophoretic analyses individually comprise capillary gel electrophoresis and capillary zone electrophoresis methodologies operating on separate microfluidic chips in different separation modules. In certain preferred embodiments, the electrophoretic separations are operated in a constant-current mode as provided elsewhere herein.

Certain embodiments of the processes of the present invention also include detecting at least a portion of the separated proteins on the microfluidic chip using a laser-induced fluorescence detector. In these embodiments, the detecting step generates one or more signals that correlate to the concentration and separation time of the separated proteins. The processes further include analyzing the one or more signals for identifying viruses. Signal analysis for identifying the virus is typically conducted by correlating the concentration and said separation time to a viral protein signature, as provided elsewhere herein.

Various embodiments of the processes of the present invention also may include preconcentration of the biological agents on the microfluidic chip. In various embodiments of the processes of the present invention, preconcentrating of solubilized proteins is typically conducted on a microfluidic chip by providing a solution comprising solubilized proteins and ions in a first channel residing in the microfluidic chip, and conducting ions from the first channel through a porous surface to a second channel residing in the microfluidic chip. Without being bound by a particular theory of operation, the transport of ions out of liquid sample in the first channel increases the concentration of the solubilized proteins in the first channel by attracting the proteins to stay proximate near the porous surface. Typically, the first and second channels are provided proximate to each other with the porous surface spanning the top portion of the microfluidic chip and in contact with each of the channels as provided elsewhere herein. Ions are typically conducted from the first channel through the porous surface to the second channel by action of a suitable fluid conducting field. Suitable conducting fields typically include a pressure field or an electric field. Suitable pressure fields may include components derived from a hydrostatic pressure, a hydrodynamic pressure, an osmotic pressure, a surface tension, or any combination thereof. Suitable pressure fields can be effected by a suitable selection of materials and pressures.

In another embodiment of the present invention there is provided a process that identifies a chemical or biological agent by generating component signatures of an unknown sample and correlating the generated component signatures to a library of known component signatures. This embodiment is particularly useful for identifying biological agents that include a biotoxin, a bacterium, a virus, a nucleic acid, a portion of biotoxin, a portion of a bacterium, a portion of a virus, a nucleic acid, or any combination thereof. This embodiment typically includes solubilizing components of sample containing a suspected chemical or a biological agent, optionally preconcentrating the solubilized components according to any of the preconcentrating methods described elsewhere herein, and labeling at least a portion of said solubilized components with a fluorescent dye to provide labeled components. The labeled components are typically injected electrokinetically into at least one microchannel electrophoretic separator, and the labeled components are separated electrophoretically using a controlled electric field. In this embodiment, the controlled electric field is preferably operated in a constant-current mode as provided elsewhere herein. The separated components are detected by using a laser-induced fluorescence detector. Suitable detectors generate signals that are correlated to the concentration and separation time of the labeled components. The concentration and separation time information of the labeled components are used to generate an unknown agent component signature. This signature is correlated to signatures stored in a database for identifying the chemical or biological agent.

In another embodiment of the present invention, there is provided a process that is particularly useful for identifying a chemical agent or biological agent isoform among the individual agent component signatures. In this embodiment, the process typically includes individually solubilizing components of at least two samples comprising a chemical agent, a biological agent, or both, to provide solubilized components. Suitable biological agents that can be identified using this process typically includes a biotoxin, a bacterium, a virus, a nucleic acid, a portion of biotoxin, a portion of a bacterium, a portion of a virus or any combination thereof. The solubilized components are individually labeled with a fluorescent dye and individually injected electrokinetically into at least one microchannel electrophoretic separator. One or both of the samples may be preconcentrated using any of the preconcentration methods described elsewhere herein. The labeled components are then individually electrophoretically separated using a controlled electric field operating in a constant-current mode to provide separated components. The separated components are individually detected by using a laser-induced fluorescence detector. Typically, the detector generates signals that are correlatable to the concentration and separation time of the labeled components. An agent component signature composed of the concentration and separation time information is individually generated for each of the samples, and a chemical agent or biological agent isoform is identified among the individual agent component signatures.

In another embodiment of the present invention there are provided processes for identifying the identity of a biological entity by analyzing its macromolecular signature. Typical macromolecular signatures typically include the spectrum of amino acids, nucleic acids, or both, that are commonly found in biological entities. In these processes, a sample including macromolecules derived from a biological entity is provided and at least a portion of the macromolecules are solubilized to provide solubilized macromolecules. At least a portion of the solubilized macromolecules are labeled with a fluorescent dye to provide labeled macromolecules. Optionally, the solubilized macromolecules are preconcentrated using a suitable preconcentration technique as provided elsewhere herein. At least a portion of the labeled macromolecules are electrokinetically injected into a microchannel electrophoretic separator. The labeled macromolecules are electrophoretically separating using a controlled electric field operating in a constant-current mode to provide separated macromolecules. The separated macromolecules are detected using a laser-induced fluorescence detector. The detector generates signals that are correlated to the concentration and separation time of the separated macromolecules. A macromolecular signature composed of the concentration and macromolecular separation time is generated using a suitable data processor. The macromolecular signature is subsequently analyzed and compared to a database of macromolecular signatures to identify the biological entity.

Various systems of the present invention can be configured for small molecule analysis as well as macromolecule (e.g., protein) analysis. Generally, the systems are handheld chemical analysis systems that combine sample handling, separation, and detection. The systems are typically capable of combining three cascaded stages; each realized using microfabricated components. Stage one collects and optionally concentrates samples. Stage two separates samples into its molecular components. Stage three detects the presence of molecular components and generates signals for data processing for sample identification.

When configured for macromolecular analysis, stage one of certain systems of the present invention collect and optionally concentrates samples. Stage two achieves sample separations, such as using capillary gel electrophoresis (CGE) or capillary zone electrophoresis (CZE) for separating macromolecules. Stage three includes a detector, such as an array of surface acoustic wave (SAW) sensors used to detect small molecule samples, or laser-induced fluorescence (LIF) detection for detecting fluorescently-labeled molecules (e.g., proteins).

In various embodiments of the present invention, the first stage of the systems optionally include a preconcentrator. A preconcentrator is a sample collection/concentration stage that samples and collects analytes from an inlet sample stream and ejects them on command into the separation stage. Sample preconcentration is typically carried out by selectively trapping analytes of interest while filtering out unwanted contaminants. In one embodiment, the preconcentrator includes a porous material in which sample fluid impinges, which results in the collection of macromolecules near the surface of the porous material while passing smaller molecules (e.g., ions) through the porous material. Any type of porous material capable of blocking macromolecules and passing small molecules therethrough is suitable, which typically includes porous membranes, and porous surfaces. Examples of porous surfaces include a cover material bonded to a rough surface. By judicious choice of this preconcentrator porous material, macromolecules of interest can be captured, while allowing small molecules to pass through, which results in preconcentration of the macromolecules. Typically, the preconcentrator is positioned between the sample inlet and the separation microchannels. Trapped analytes of interest can be released by adjusting the applied electric field, temperature or ionic strength of the solution, such as the salt concentration. When this environment is altered with in the preconcentrator the analytes are then redirected to the separation capillary for separation in the microchannel. In this fashion, the concentrated macromolecules on the porous material are swept towards the separator as a result of this change in potentials.

Just about any biological entity can be analyzed according to the present invention. Typically, the set of identifying molecules of the biological entity can be separated using electrophoresis techniques. Suitable biological entities include prokaryotic such as viruses, bacteria as well as eukaryotic life forms, and, plants. Biological substances synthesized by such life forms are also included in the set of biological entities that are identifiable according to the present invention, examples of which include biotoxins.

Systems of the present invention are preferably designed to support current control by providing rapid current measurements and updating high voltages. Typical modular high voltage power supplies can be used that can generate a full-scale stepped voltage in 20 milli seconds (“ms”), with current measurements of equal speed. A microprocessor is typically provided for reading at least one and preferably at least 12 currents and updates at least one and preferably at least 12 voltages at least every 100 ms, and preferably at least 50 ms. Preferably such microprocessors are embedded in the power supplies.

To accommodate current control, software for the microprocessors preferably provide menu code that includes user variables and a proportional digital control routine. A proportional routine provides simple and stable current control with acceptable offset error. In modifying voltage-control microchannel flow to constant-current control multichannel flow, the event scheduling is typically changed. In voltage-control mode, the high voltages are typically updated infrequently during an injection or separation. The timer interrupt section in the voltage-control mode, accordingly, does not to require the digital-to-analog (DAC) update routine. Because the current control feedback algorithm depends on rapid updates to the high voltage as well as rapid current measurements, the DAC routine is typically coupled to the timer interrupt where the analog-to-digital (ADC) current reading routine is typically found. The LCD update routine is also typically coupled to the interrupt during runs to be with the DAC routine. In preferred embodiments, the DAC and LCD hardware share the same serial bus so that one typically does not have a higher priority than the other. The DAC routine preferably is rewritten from C programming language into assembly language for faster execution. This affords the use of lower CPU clock speeds.

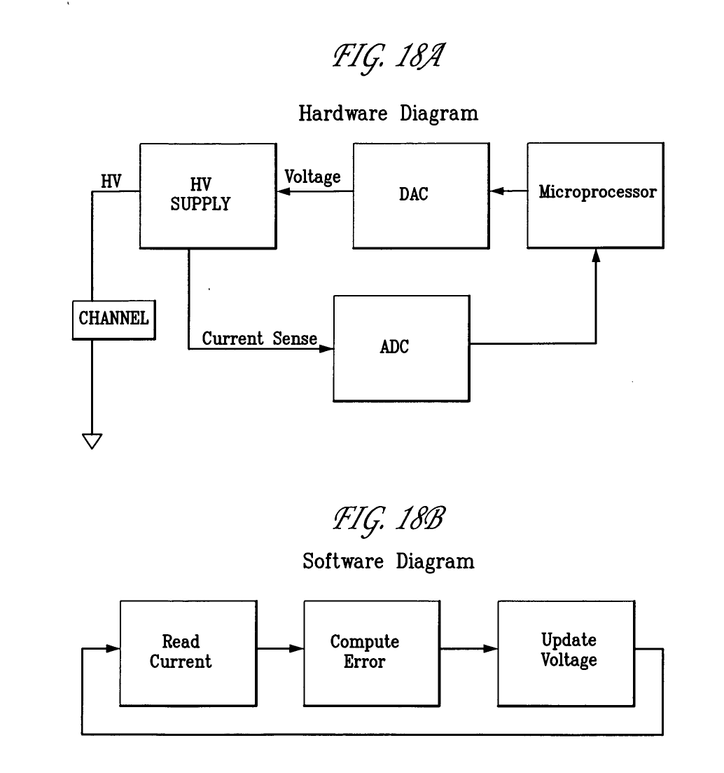

A constant current mode is typically provided as a feedback loop, as described herein. Diagrams of the hardware and software design for one embodiment of the current control procedure are provided in FIGS. 18A and 18B. Charged solutes will typically migrate through the separation channels containing a separation media, such as a polymeric material (typically an entangled polymer) as a result of the electron current flow. When the system is set up on a constant voltage (the preferred operational mode), the electron current flow (on the order of micro amperes, μA) in the channel fluctuates. These fluctuations are due to many factors including local dilutions of entangled polymers with sample buffer solution, temperature, particle contaminants, among other known chemical physical phenomena driving compositional fluctuations in condensed matter systems. Accordingly, when the current fluctuates the migration slows down (reduced current) or speeds up (increased current). To make the system more robust and less sensitive to environmental effects, a feedback loop for establishing a constant current mode of operation is provided in which the voltage is varied by the feedback loop. Determining an optimal current for operation is obtained as follows. A first separation using the system is run in a constant voltage mode to determine the optimal current setting. The system is then run in constant current mode, and voltage is varied. As shown in the results for the Example, “Detection of Viral Signatures” provided below, the constant current mode performs separations with dramatically improved reproducibility.

In one embodiment of the present invention, the programming code for the data analysis software provides a report of the molecular weight of any detected peak using capillary gel electrophoresis analysis. In reporting molecular weight, the data analysis software typically refers to a cubic fit standard curve that is generated from proteins of known molecular weight and concentration. Details are described further below.

In one preferred embodiment, identification of biotoxin variants and viral signatures are carried out using a hand portable system. In this embodiment, the hand portable system is used to rapidly detect and identify chemical, biotoxin and viral agents in the liquid phase. This embodiment uses parallel electrophoretic analyses combined with a highly sensitive laser-induced fluorescence detector that are integrated at the microchip scale. Further details are provided in the following examples.

As described herein, various systems of the present invention are designed to integrate various components into a small and robust package. Preferably, the components are accessible and interchangeable. The liquid solutions used for manipulating the proteins are typically held in reservoirs. The system design preferably provides reproducible and quick chip removal and installation. Various systems of the present invention also include short-to-ground resistant electronics. The microfluidics portion of the systems of the present invention suitably includes a small volume filtered injection port. The microfluidics portion of the system is also typically capable of providing buffer solution replacement in seconds. Also, the microfluidic fluorescence detector components are typically designed using simple, dye-free laser alignment. Such dye-free laser alignment designs typically provide easy maintenance of the optical system. The materials of construction of the system are typically selected to be compatible with solutions, electronics and optics.

### EXAMPLES

Preparing Microfluidic Chips

Microfluidic chips were generally fabricated from Corning 7980 fused silica wafers (100 mm diameter, 0.75 mm thickness using standard photolithography, wet etch, and bonding techniques. Fused Silica wafers were PECVD deposited with amorphous silicon (150 nm), which served as the hard mask. A 7.5-micron thick layer of positive photoresist was spin-coated and soft-baked (90° C., 5 minutes). The mask pattern was transferred to the photoresist by exposing it to UV light in a contact mask aligner. After exposure, the photoresist was developed and hard-baked (125° C., 30 minutes). Exposed silicon was etched in a plasma etch tool. Silicon etch process typically consisted of a 30 second oxygen ash @200W DC @25 mTorr, followed by 150 second SF6 @200W DC & 50 mTorr. The exposed glass was etched with a 49% HF solution. Via access holes were drilled in the cover plate (Corning 7980) with diamond-tipped drill bits. The etched wafers and drilled cover plates were cleaned with 4:1 H₂SO₄:H₂O₂ (100° C.), de-stressed with 1% HF solution, then the surfaces were treated in 80° C. 40% NaOH, rinsed in a cascade bath, followed by a spin rinse dry, aligned for contacting, and thermally bonded at 1150° C. for five hours in an N₂-purged programmable muffle furnace. The standard chips were cut with a programmable dicing saw containing a diamond composite blade into 25.4×25.4 mm or 20×20 mm devices depending upon design.

Example of a Hand-Portable System

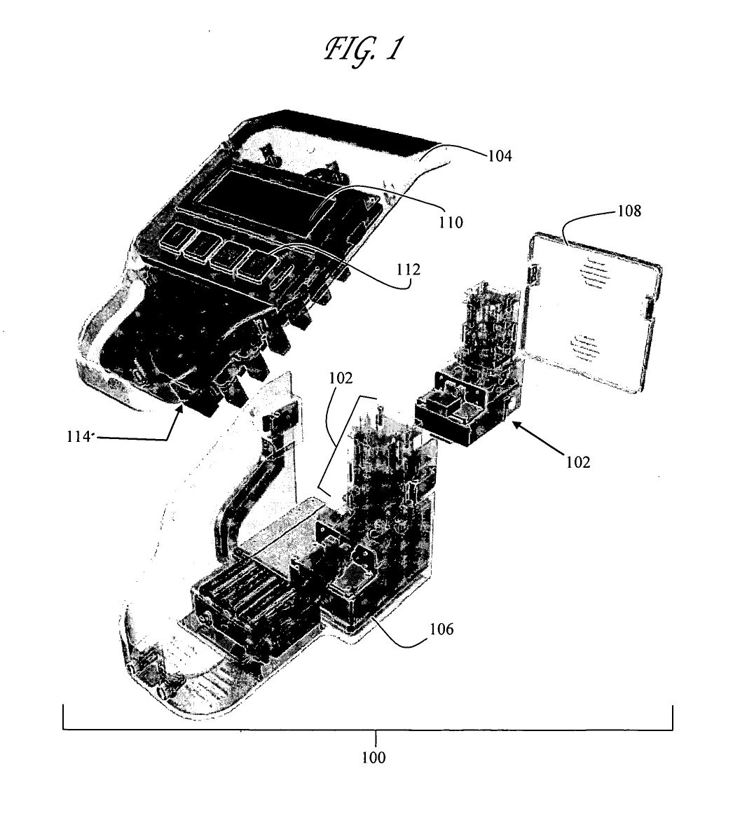

In this example, a hand-portable system was constructed to detect a broad range of chemical, biotoxin and viral agents in liquid samples. An exploded view of this hand-held microanalytical system (100) is illustrated in FIG. 1, which shows a housing having a top housing portion (104), a bottom housing portion (106) and a back plate (108), a display (110) and keypad (112) contained within the top plate (104), a high voltage board carrier (114) mounted underneath the top plate (104), two integrated microfluidic and fluorescence detector modules (102) capable of being situated next to each other on the bottom housing portion (106), and a vented back plate (108) for enclosing the system.

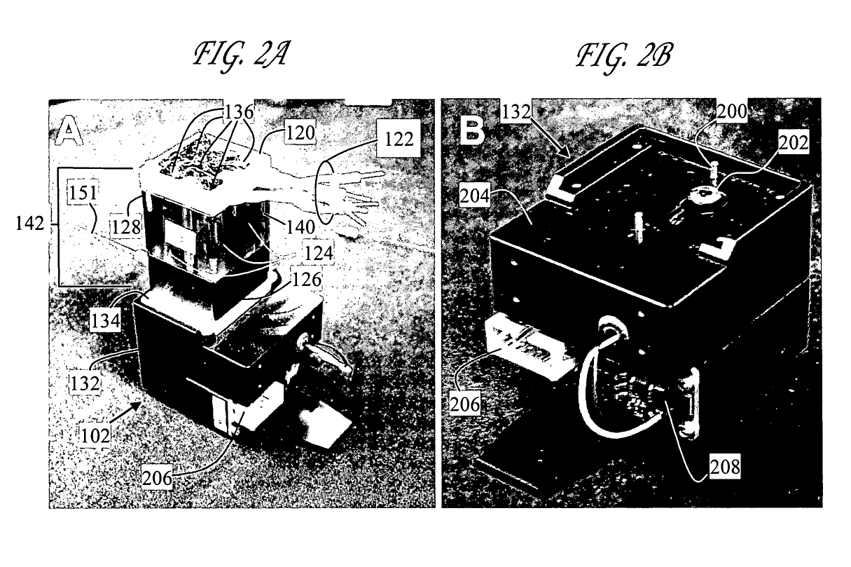

Examples of various components of the system of the present invention are illustrated in FIGS. 2A, 2B, 3 and 4. FIG. 2A illustrates a separation module (102). The separation module (102) includes a fluid cartridge (128) having reservoirs (124). The reservoirs (124) are typically individually housed in the fluid cartridge (128) containing running solutions (not shown). Below the electrode plate connector (120) are electrodes (not shown) in the reservoirs (124). The electrodes (not shown) connect buffer solutions in the reservoirs to a high voltage source through electrode plate connector (120) via electrical leads (122). The fluid cartridge comprises a bottom housing portion (140) that is connected to liquid manifold (126). Shown also is the liquid manifold (126) connected to compression frame (134), which in turn, resides atop a detector module (132). The electrode plate connector (120) is shown, attached from the fluid cartridge (128), having electrical contacts (136) and electrical leads (122). Also shown is an injection conduit (151) connected through injection port hardware (not shown) for receiving pressure injected fluid samples into the liquid manifold (126).

FIG. 2B shows a microfluidic fluorescence detector module (132) (cover on) used with the system. Shown are alignment pins (200) for aligning the compression frame (134) of the microfluidics separation module (102), an observation lens (202), a detector cover (204), and a scalable laser/PMT board (208). The detector module (132) uses a single connection (206) to the main board of the system (100) (not shown). The dimensions of the detector module (132) were approximately 7.5×5.5×3 cm. The detector incorporated rapid (typically requiring less than about five minutes), dye-free, alignment of the laser optics.

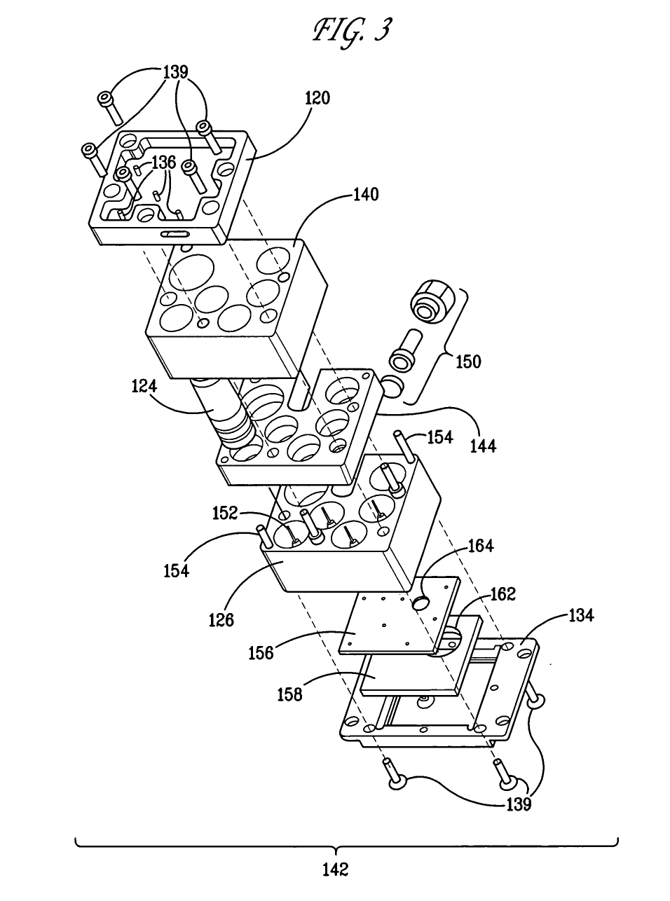

FIG. 3 illustrates an exploded view of the microfluidic portion (142) of the separation module (102) depicted in FIG. 2A. The electrode plate connector (120) connects a high voltage source (not shown) through electrical contacts (136) to running buffer solutions contained within reservoirs (124) (one shown). The electrode plate connector (120) is held to the top portion of the fluid cartridge housing (140) by way of screws (139). Capillaries (152) in the liquid manifold (126) provide liquid connection between the reservoirs (124) and the microfluidic chip (156). The alignment pins (154) align placement of the liquid manifold (126) with the bottom portion of the fluid cartridge housing (144). The compression frame (134) is held against the compression plate (158), which is held against the microfluidic chip (156) for fluidic sealing to the liquid manifold (126), all of which is held together by screws (139). An opening (162) in the compression plate (158) provides for placement of an observation lens of the detector module (not shown, described further below) close to the detection region (164) in a microchannel separator (not shown) on the microfluidic chip (156). Also shown is the relative placement of injection port hardware (150) for the liquid manifold to receive pressure injected fluid samples.

FIG. 4 depicts the underside of the microfluidic portion of the separation module (142) shown in FIG. 3 by viewing the compression frame (134). Depicted are o-ring face seals (160) (shown as dotted lines beneath the microfluidic chip) that enable simple chip installation in the microfluidic portion of the separation module (142). The opening (162) in the compression plate (158) provides for placement of an observation lens of the detector module (not shown, described further below) close to the detection region (164) of a separator channel (not shown) on the microfluidic chip (156). The compression frame (134) is held against the compression plate (158), which is held against the microfluidic chip (156) for fluidic sealing to the liquid manifold (126), held together by screws (139). As illustrated, PF indicates pressure injected samples entering the chip via a face seals (160). Pressure injected samples are then electrokinetically injected by applying voltage between the sample (S) and sample waste (SW) channel (further details of electrokinetic injection are described and shown in FIG. 8, below). To perform reducible sample injection, injection current on the buffer leg is preferably held constant. After injection, separation is performed by applying a controlled, constant current from buffer (B) to waste (W) for electrophoretic separation through the separation channel. Examples of suitable channel voltages and/or currents for injection and separation are provided in the following table.

| Channel | Injection | Separation |
|---|---|---|
| Sample (S) | 0 V | 450 V |
| Sample Waste (SW) | 900 V | 450 V |
| Buffer (B) | 0.6 μA controlled | 0 V |
| | current (@ 400 V) | |
| Waste (W) | 450 V | 11.0 μA controlled |
| | | current (@ 4500 V) |

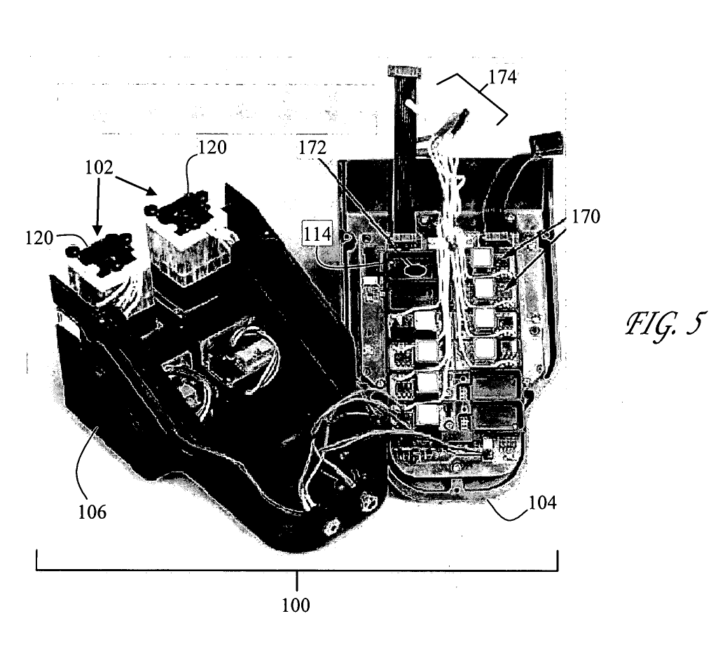

FIG. 5 illustrates one embodiment of the system (100) (opened, back plate not shown) of the present invention having two separation modules (102) that reside side-by-side in the bottom housing portion (106), and a high voltage board carrier (114) mounted in the top housing portion (104). The high voltage board carrier (114) is depicted as having 12 channel carrier slots (172), four of which are empty. Also depicted are high voltage supply leads (174) from each of the high voltage boards (170), each of which are disconnected from the electrode plate connectors (120) of the two separation modules (120). The 12-channel high voltage design supports any electrically driven experiment. As described further, the high voltage supply was designed to provide high voltage up to ±5 kilovolts (“kV”) at less than 100 microamps (μA). The voltage supply typically provided precision electrical current monitoring and voltage control using digital-to-analog (“D-A”) interfacing and embedded central processing unit (“CPU”) control. The high voltage supply was short-circuit protected.

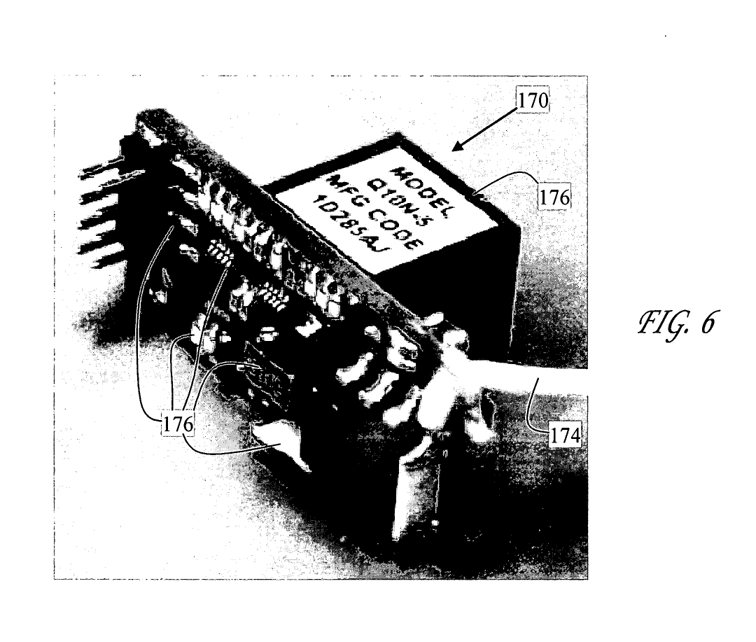

FIG. 6 depicts one of the high-voltage (HV) boards (170) shown in FIG. 5 with electronic components (176) and a high voltage lead wire connected thereto. The modular design of the HV board enables it to be plugged into any of the 12 carrier slots (172) of the HV board carrier (114) of FIG. 5. Typical dimensions of the high voltage boards (170) are about 15 mm×30 mm×15 mm.

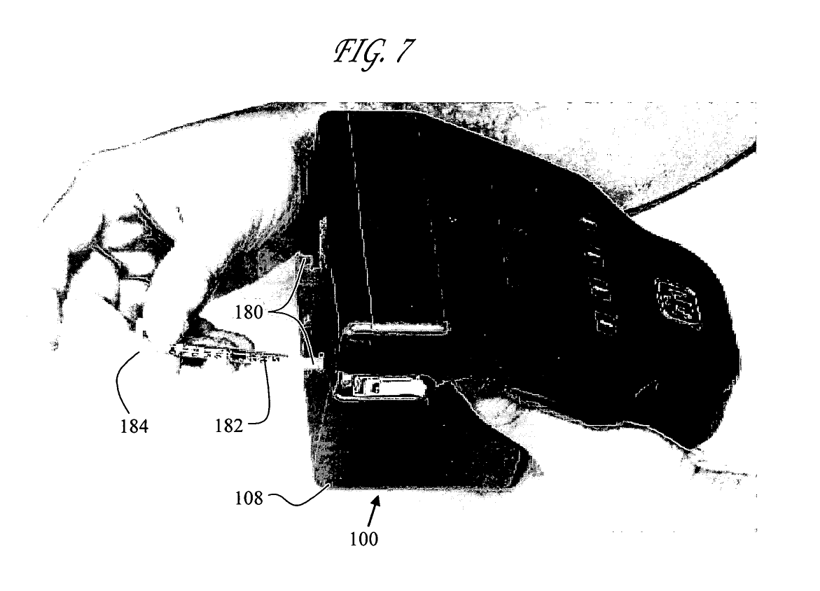

FIG. 7 shows microfluidic injection of a liquid sample (182) into one of the injection ports (180) into one of the separation modules (not shown within the housing) of one system (100) of the present invention. Samples are typically injected using a syringe (184) through the back plate (108) of the system (100). The injection ports (180) are fluidically connected to injection conduit (151) of the separation module (not shown, within the housing).

FIG. 8 illustrates integration of pressure and electrokinetic injection on the microfluidic chip (156). Liquid sample (182) is pressure injected into holding channel (190). Shown using arrows, the liquid sample is electrokinetically injected from the holding channel (190) through EK injection channel (230) into the offset tee (“offset T”) sample loop (196) that provides a sample injection plug (194). The offset T is formed by the fluidic combination and positioning, as shown, of the EK injection channel (230), the separation channel (192) and the waste channel (198). The system was provided with microfluidic chip-based CGE and CZE micro separations and LIF detection for analyzing proteins in liquid samples. On-chip sample preconcentration was provided using the procedures described herein and the microfluidic chips depicted in FIGS. 19 and 20.

General Procedures—Microchannel Chip Design and Operation

FIG. 4 shows injecting of a sample through a liquid manifold onto a microchannel chip. Samples are pressure injected using a syringe fitted with a threaded connection that mates to the injection port in the rear of the hand-held dual channel protein separations instrument (FIG. 7) which contains a fluidic manifold (B). Pressure injected samples enter the microfluidic chip via a face seal connection (PF) onto a 2.0 cm² fused silica microfluidic chip. Pressure injected samples are then electrophoretically injected by applying voltage between the sample (S) and sample waste (SW) channel. To perform reducible sample injection, injection current on the buffer leg was typically held constant. After injection, separation is performed by applying an 11.0 μA constant current from buffer (B) to waste (W) for electrophoretic separation through the 10 cm separation channel.

FIG. 11 shows the layout of a microfluidic chip (156) used in these examples. FIG. 11 indicates the locations of the electrodes that control the potentials (voltages, currents) and the reservoirs (depicted as circles) in relation to the chip components and ports. The microfluidic chip (156) includes a sample reservoir port (232) for receiving liquid sample from the reservoir that contains electrode S (not shown). The sample reservoir port (232) is in fluid communication with holding channel (190) that is in fluid communication with sample injection port (234), for pressure flow injection, (“PF”). Sample injection port (234) is also in fluid communication with electrokinetic injection channel (230). As shown, the electrokinetic injection channel (230) is typically much narrower than the holding channel (190) to effect hydrostatic isolation of these two channels. Electrokinetic injection channel (230) terminates at separation channel (192) to form an offset T loop (196) with waste channel (198). Waste channel (198) terminates at the sample waste port (242) for delivering sample waste liquid to the waste reservoir that contains electrode SW (not shown). Also shown in FIG. 11 is flush port (236) through which separation media (such as PEO gel) from a separation media reservoir (not shown) fills separation media fill channel (244). Buffer reservoir port (240) is in fluid communication with separation channel (192) and with a buffer reservoir (not shown). The separation channel (192) is typically filled first with buffer using pressure injection of buffer through buffer reservoir port (240), into the separation channel (192), and into waste reservoir port (238). After the separation channel is filled with buffer, fluid separation media residing in the separation fill channel (244) is electrokinetically transported into separation channel (192) by application of a potential between an electrode in the waste reservoir (W, not shown) and an electrode in the reservoir containing the separation media (not shown). Location of the detection window (164) is also indicated.

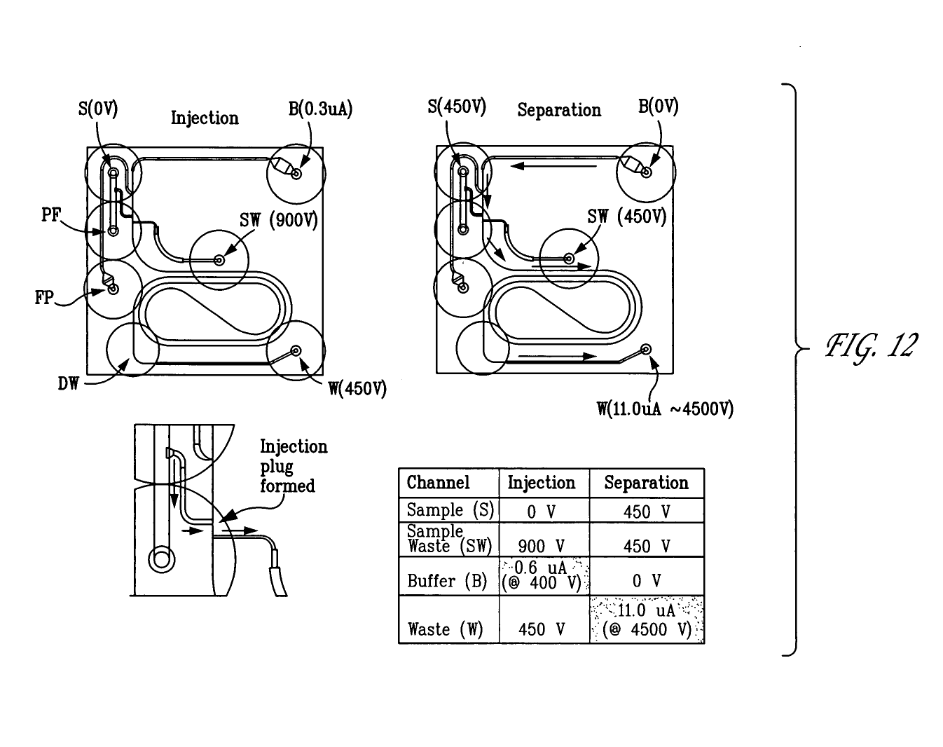

FIG. 12 indicates the potentials (voltage or current) among the microchannels during injection and separation, the arrows indicating the direction of the analyte movement (e.g., electrophoretic flow). In this example, an EK injection was performed after pressure injection (FIG. 12, lower left, diagram depicting movement with arrows, annotated as “Injection plug formed”). The current flow and the charged species moved from the sample electrode (S) to the sample waste (SW) electrode, as indicated by the arrows, on a 900 V potential between these electrodes (upper left diagram). During this injection, sample plug shape was maintained by applying a voltage between the buffer (B) and waste channels. A controlled current on the buffer (B) channel typically helped maintain equal current flow between the sample (S) and sample waste (SW) channels. After a specified time, the injection was ended and the separation mode was started. In this mode, voltages up to about 10 kV were applied between the buffer channel and the waste channel. Separations were carried out using constant current of 11.0 μA (@450V/cm) on the waste leg. During separation mode, the sample (S) and sample waste (SW) leg voltages were controlled to minimize the injection of additional sample. Charged analytes typically moved through the separation channel, as indicated by the arrows in FIG. 12 (upper right diagram). All other channel voltages were set to optimize the injection/reduce sample carryover. Separations were performed on a fabricated 2.0 cm² fused silica chip, with a separation channel filled with a polyethylene oxide/polyethylene glycol protein separation gel (Beckman, Fullerton, Calif.). Typical separation channel lengths typically were about 10 cm, but microfluidic chips having separation channels as long as about 30 cm have been used. The injected sample plug eventually separates to form distinct analytes bands which are detected as specific peaks at the detection window (DW). Detection was accomplished by epi-fluorescent imaging of the separation channel using a ultraviolet laser diode (Nichia, Tokushima, Japan) and supporting optics (excitation 395 nm/emission 460 nm). Detected peaks were routed through an A/D converter and either saved on the system's data processor for later analysis or viewed directly via a serial port connection on a laptop computer running Labview™ software (National Instruments, Austin, Tex.).

### EXAMPLE

### Identifying Macromolecular Specimens

The hand-portable system was provided to operate as a liquid-phase microanalytical instrument for analytically separating and identifying macromolecular specimens. The system included the following components and operating characteristics: multiple orthogonal separation methods running simultaneously; pressure injection of samples; laser-induced fluorescence detection of sample analytes; hand-portable, stand-alone, on-board data collection and analysis; low-power consumption, battery-operated; and an instrument platform capable of accommodating a wide-variety of microseparation methods. The system had few hardware failures, required minimal downtime during component replacement, operated reliably, and had good sensitivity. Results using this system are described below.

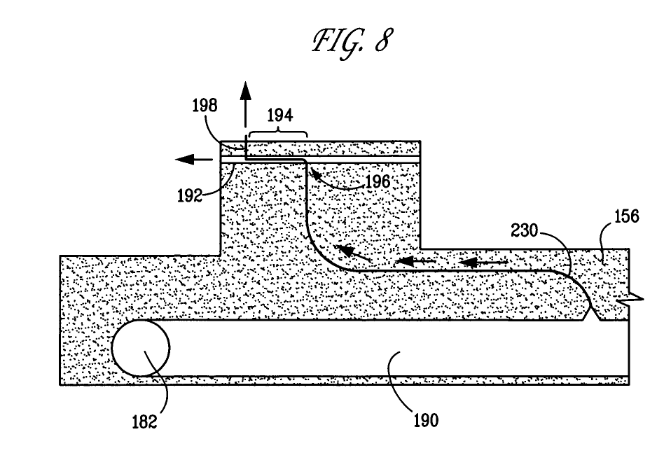

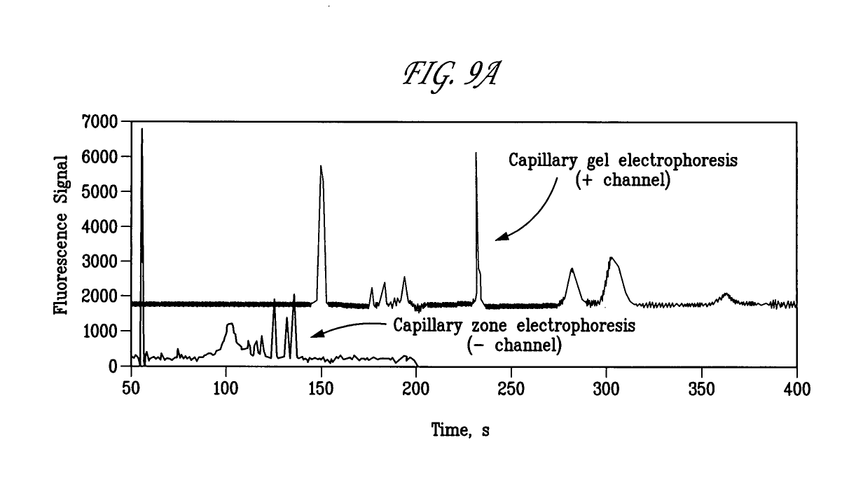

FIG. 9A depicts the simultaneous electrophoretic separations of a protein sample using four positive (top—capillary gel electrophoresis) and four negative (bottom—capillary zone electrophoresis) high voltage channels using this system. Analysis of protein standards with known molecular weights (CGE analysis) and protein charge or pI (CZE analysis) show that dual channels orthogonal separation techniques can be performed using the system described herein. In this example CGE analysis was performed on HPTS, a small fluorescent molecule, cytokinin peptide (CCK), α-lactalbumin (lact), carbonic anhydrase (CA), ovalbumin (OVA), and bovine serum albumin (BSA) along with CZE analysis of CCK, Lact and OVA.

FIG. 9B depicts the improvement seen in using on-chip sample preconcentration and separation of a protein sample (20 nanomolar (“nM”) Lactalbumin, 20 nM Ovalbumin) using six positive high voltage channels. Top—60 second preconcentration of the protein sample. Bottom—no preconcentration of the protein sample. The results indicate that dilute protein samples can be concentrated in microchannels as much as 100 fold prior to electrophoretic sample analysis.

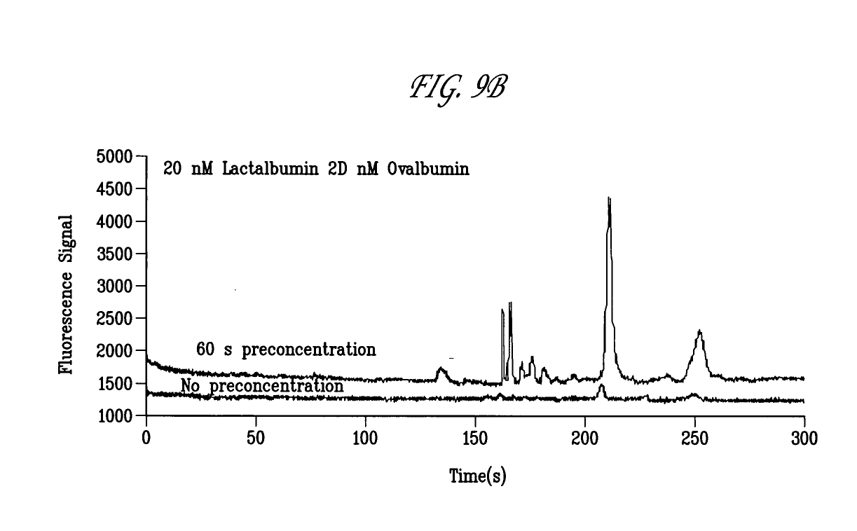

FIG. 10 depicts separation of fluorescamine labeled ricin biotoxin. Top trace—600 pM sample injected; bottom trace—300 pM sample injected. These results indicate that proteins can be detected in the picomolar range, without preconcentration, using the systems and methods of the invention.

### EXAMPLE

### Use of Constant Current Separations to Enable the Determination of Molecular Weight of Proteins

The system described in “Example of a Hand-Portable System”, above, was operated in a constant current mode in this example to carry out viral proteomic analysis. A separation technique was developed to provide separation times having an improved tolerance for time error, giving rise to molecular weight measurements with improved accuracy. Sample introduction methods that can reproducibly inject essentially the same amounts over an entire day without flushing were also developed. As described herein, both software and hardware of a handheld system were improved to allow the injection and separation to be completed while maintaining constant current and minimizing voltage fluctuations to optimize injection and separation performance. Injection reproducibility was typically improved by combining pinch voltages and constant current control. In these experiments, a positive 900 volt potential was applied between sample waste and the sample arm (e.g., FIG. 4). Controlling the buffer leg voltage during EK injection typically controlled sample plug formation. A 1:1 voltage ratio between sample and sample waste was maintained by altering the buffer channel to remain at a constant current. Applying a slightly positive current (reverse flow-towards the buffer leg) typically provided the greatest intensity of plug formation without compromising peak shape during separations. This was confirmed by imaging the sample plug formed with fluorescein isothiocyanate (FITC)-labeled proteins (data not shown). Applying constant current control to separations also improved measurement reproducibility. When separation of standards of proteins consisting of cytokinin peptide (CCK), α-lactalbumin (lact), carbonic anhydrase (CA), ovalbumin (OVA), bovine serum albumin (BSA) and immunoglobulin G (IgG) of known molecular weights were carried out under constant voltage control (FIG. 14A), some run-to-run drift in separation times was apparent. Separation times appeared to drift approximately 5% over a few hours. In contrast, separation times were more steady, less prone to drift, and more reproducible when the proteins were separated using constant current control mode, during which the voltage was regulated to achieve constant current. Using the constant current technique reduced the relative standard error between runs (FIG. 14B). Uncorrected data sets had a relative standard error between runs (n>7) as low as 0.2%, and were usually below 1%. (FIG. 14C) The reproducibility was not affected by protein size, as larger proteins had approximately the same error associated with separation of smaller proteins. Results similar to these were collected over a series of days for multiple chips. While run-to-run data for different chips/days had similarly low errors associated with the separation, chip to chip reproducibility was marginally higher (ca. 3%). Chip to chip reproducibility was typically improved using a standard calibration when replacing the chips.

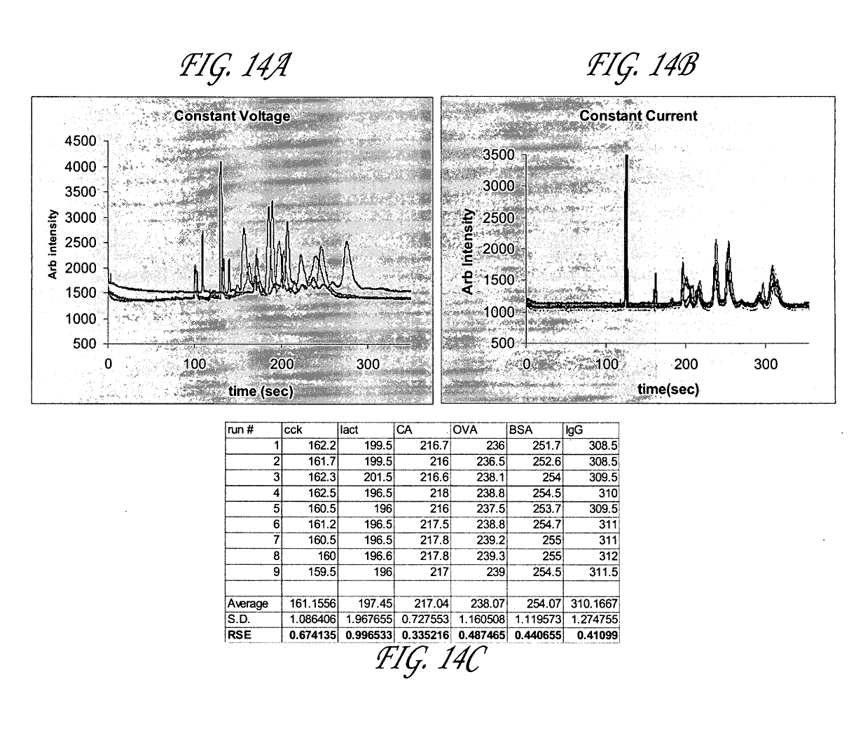

Separations were typically carried out in 100% Sieving gel (Beckman, Fullerton Calif.). Typically, the gel was infrequently replaced. Gel replacement tended to adversely affect reproducibility, apparently due to movement in the channel after flushing. Using this constant current separation mode enabled the calculation of the molecular weights of proteins CCK, Lact, Ova, BSA and IgG, as depicted in FIGS. 16A, 16B and 16C. In addition to measuring peak pattern as a function of retention time, it was useful to obtain the distribution of molecular weights of the constituent proteins. The distribution of molecular weights typically provided a species signature that was independent of the measurement method, e.g., FIG. 16B. A calibration curve of MW vs. retention time was obtained using a set of proteins of known masses (CCK @1.1, Lact @14.2, CH @29.5, Ova @45, BSA @65 and IgG @150 kDa). The same calibration standards (HPTS and IgG) as described above were used for this measurement. FIG. 16A shows the MW as a function of the measured retention time along with a least squares fit of the data to a cubic polynomial. While this cubic polynomial is empirical, a fit that is based on a theoretical model of the gel separation process could also be used.

Based on the correspondence between retention time and MW, the distribution of MWs was determined. Although this distribution is typically discrete in nature, the axial diffusion during the separation process typically causes spreading of the peaks and thus the distribution of MWs can be approximately represented by a continuous function. To obtain the discrete distribution the isolated peaks were collapse into a series of histograms. Peaks that were broader than diffusion widths typically were not uniquely decomposed into discrete MWs, so approximate groupings for these peaks were obtained.

Without being bound by any particular theory of operation, the number of proteins passing the detection point during the sampling period is estimated using the following mathematical construct. The number of proteins passing the detection point is: ΔN=n(t) Au(t) Δt, where n(t) is the protein density as a function of retention time, A is the effective flow area and u(t) is the flow velocity. Let μ denote the MW (molecular weight) and ƒ(μ) the distribution of MWs. Given the one-to-one correspondence between MW and retention time, the molecular weight distribution can be expressed as:

$$f ( μ ) ⅆ μ = α n ( t ) A \frac{u ( t )}{{u}_{0}} ⅆ t ,$$

where α is a proportionality constant that is determined by the normalization condition on the distribution and uₒ is a reference flow velocity. Since the measured signal is proportional to n(t), one can write:

$$f ( μ ) ∝ S ( t ) \frac{u ( t )}{{u}_{o}} \frac{ⅆ t}{ⅆ μ} ,$$

where S(t) is the measured signal and dt/dμ is obtained from the cubic fit. Assuming that the flow velocity is constant for a given MW, one can write u(t)=d/t, where d is the separation distance to the detector provides:

$$f ( μ ) ∝ S ( t ) \frac{{t}_{o}}{t} \frac{ⅆ t}{ⅆ μ} ,$$

where tₒ is an arbitrary reference time. Note that the 1/t dependence comes from the fact that slower proteins have longer residence time in front of the detection laser. Using the above expression and the cubic fit the distribution of MWs are obtained for a given measured signal.

A cubic fit of MW vs. retention time based on four measurements was earlier obtained. The data plotted in FIG. 16A shows a cubic fit along with the corrected data. A new dataset was obtained after implementation of the constant current circuitry in the system. Although reduced in accuracy, it is informative to use a linear correction to correct this data and use the corrected data to predict molecular weights based on a cubic fit. Without being bound by a particular theory of operation, it appears that the reduced accuracy arises from the old data not being adequately represented by the linear correction. This reduced accuracy is possibly due to the observed peak shape changes and variable current during separation. The new dataset was linearly corrected relative to the first measurement of the old data. CCK and IgG were used as calibration standards. The following table shows raw and corrected retention times.

TABLE 1
Retention time vs. MW

| | Old data | | New data | | | Corrected | Calculated |
|---|---|---|---|---|---|---|---|
| PROTEIN | time −col1 | MW | time (day 2) | Dt | Linear (Dt) | time | MW |
| CCK | 140.07 | 1.1 | 171.96 | −31.89 | −31.89 | 140.07 | |
| Lact | 172.09 | 14.2 | 209.02 | | −36.58 | 172.44 | 12.7 |
| Ova | 207.34 | 45 | 252.29 | | −42.04 | 210.25 | 47.9 |
| BSA | 223.44 | 66 | 269.53 | | −44.22 | 225.31 | 67.1 |
| IgG | 276.82 | 150 | 328.5 | −51.68 | −51.68 | 276.82 | |

Although there were errors in the calculated molecular weights, the errors were somewhat minor. The calculated molecular weights are significantly more accurate when constant current measurements are used with a linear retention time correction to obtain the fit of molecular weight as a function of retention time.

The MW vs. retention time data was fitted using a polynomial with the following form:

$$MW = {\sum }_{k = 0}^{N} {C}_{k} {\{ \frac{t}{{t}_{o}} \}}^{k}$$

where t is the retention time in seconds and the value of tₒ used is ½ the maximum value of the measured retention time is about 138.9.

The calculated coefficients for the quadratic and cubic cases are respectively:

N = 2:

| | |
|---|---|
| k = 0 | C₀: 90.718846 |
| k = 1 | C₁: −211.563021 |
| k = 2 | C₂: 120.995093 |

N = 3

| | |
|---|---|
| k = 0 | C₀: 239.932714 |
| k = 1 | C₁: −530.275318 |
| k = 2 | C₂: 340.040394 |
| k = 3 | C₃: −48.558091 |

### EXAMPLE

### Detection of Viral Signatures

Viral signatures were generated as follows. First, bacteriophage T2, T4, and T6 were grown and purified. Phage were produced by the multi-cycle lysis-inhibition technique described by Doermann, et al. “Genetic control of capsid length in bacteriophage T4. I. Isolation and preliminary description of four new mutants.” J. Virol 12(2): 374-85 (1973). An overnight culture of the appropriate host strain was diluted 1:100 into IL medium M103 (medium M9 plus 1% casamino acids) and incubated at 37° C. with aeration until the cell density reached 4×10⁸ cfu/mL. The culture was shifted to 30° C. and cells were infected at a MOI of 0.1 pfu per cfu. Incubation continued at 30° C. for 180 minutes post-infection when virus containing bacteria were harvested by centrifligation and resuspended in 50 mL of buffer BUM (13.3 g/L Na₂HPO₄·7 HOH, 4 g/L NaCl, 3 g/L KH₂PO₄ and 1 mM MgSO₄. Cells were lysed by vortexing in the presence of CHCl₃. Cellular debris was removed by centrifugation at 3000×g for 15 minutes. Phage were pelleted by centrifugation at 18000×g for 1 hr. Phage pellets were covered with 25 mL of buffer BUM and stored overnight at 4° C. prior to resuspension by gentle mixing. The integrity and purity of these viral preparations were confirmed by transmission electron microscopy. Briefly, virus were diluted in water and placed on gold grids (Ted Pella, Reading, Calif.). Samples were then briefly negatively stained with phospho-tungsten and immediately dried. Dried stained phage virus was then imaged on a Zeiss EM-10 transmission electron microscope at 60 kV. Magnification was calibrated by imaging a 200 nm grid.

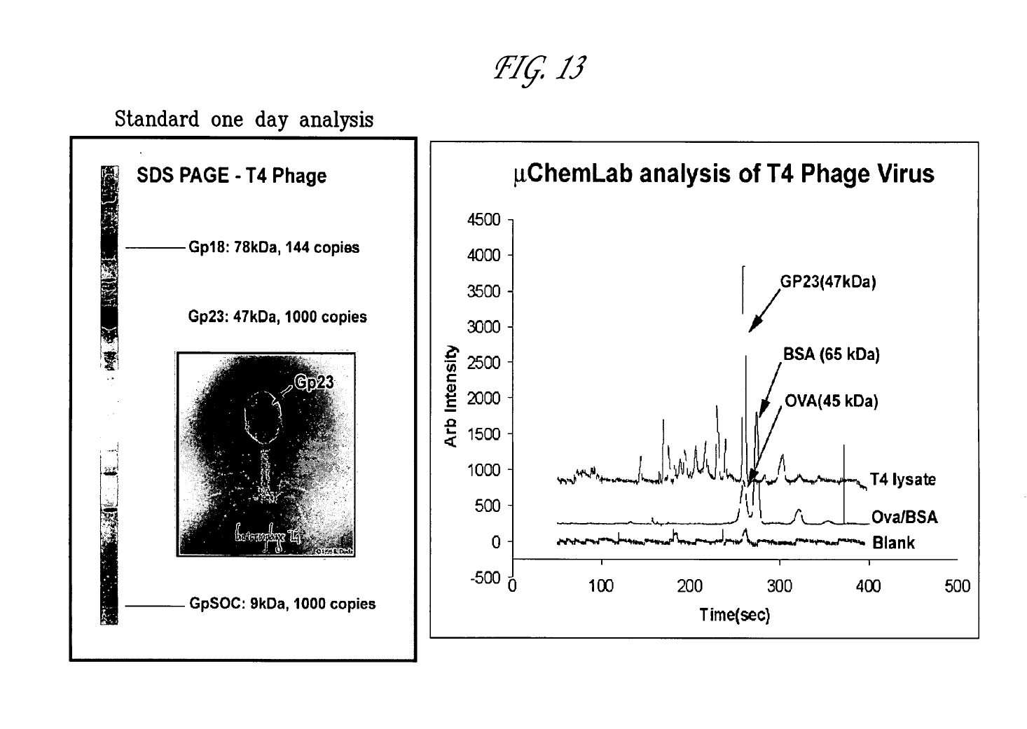

Images of these viral preparations demonstrated that bacteriophage had typical structure, as seen in FIG. 13. Bactriopghage T2, T4, and T6, all had indentical structures as judged by electron microscopy. These viral particles had a isodecahedral viral capsid head, with a narrow tail extending downward to fine leg like structures (FIG. 13). In some cases, viral ghosts were present. These viral particles had injected their capsid contents, and appeared to have a shorter tail structure with leg folded upon the capsid head. These samples were regrown and purified to ensure integrity of the viral preparation.

Upon confirmation of viral stock solution integrity, signatures were obtained by diluting purified stocks 1:40 dilution in a lysis buffer containing 5 mM boric acid, 5 mM sodium lauryl sulfate in water drop-wise adjusted to pH 8.5 with 1M NaOH. Diluted samples were placed on a heating block set at 95° C. for five minutes. After samples were removed from heat, 10 mM fluorescamine dye was added (1 mM working concentration), and samples were vortexed. Fluorescamine labeled virus samples (15 microliters (“μl”)) were then pressure injected into the microfluidic system. After pressure injection of the fluorescamine labeled viral proteins an EK injection was performed followed by a electrophoretic separation of the viral coat protein analytes.

CGE analysis of purified bacteriophage generated a characteristic electropherogram of solubilized viral particles (FIG. 13) based on molecular weight of protein species present. Electropherograms or signatures of solubilized viral particles demonstrated a high abundance of the high copy number protein GP23, a 47 kDa viral capsid protein, along with the detection proteins of lesser abundance (FIG. 13, right). Signatures of viral proteins demonstrated good correlation to standard slab gel electrophoresis performed of viral stocks (FIG. 13, left). To ensure that consistent and reproducible signatures were generated, separation of viral proteins were carried out using constant current CGE analysis (FIGS. 15A, B, C). Constant current analysis generated signatures provided robust signature development as judged by migration time of detected protein species (FIG. 15A). Run-to-run migration time of selected protein species were analyzed and found to low variability and low migration time error with relative standard error typically less than 2% (FIGS. 15B, C).

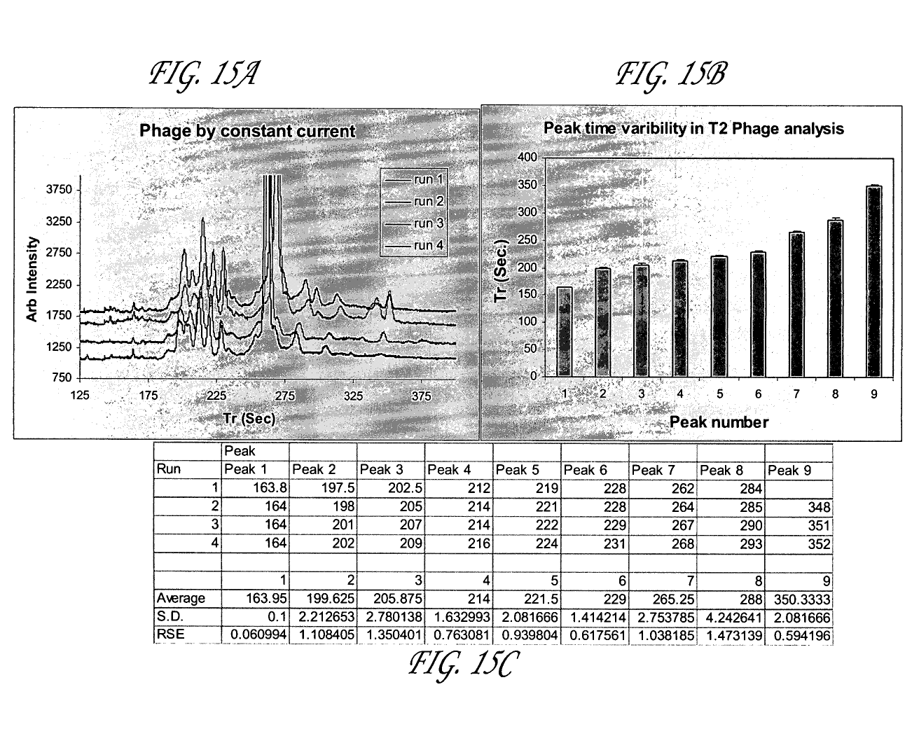

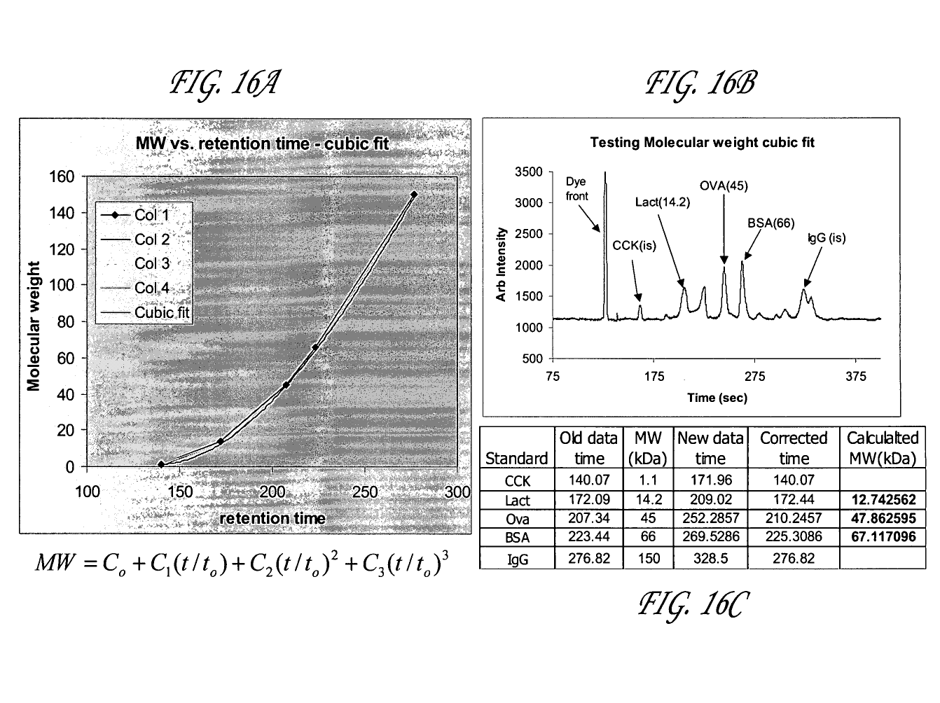

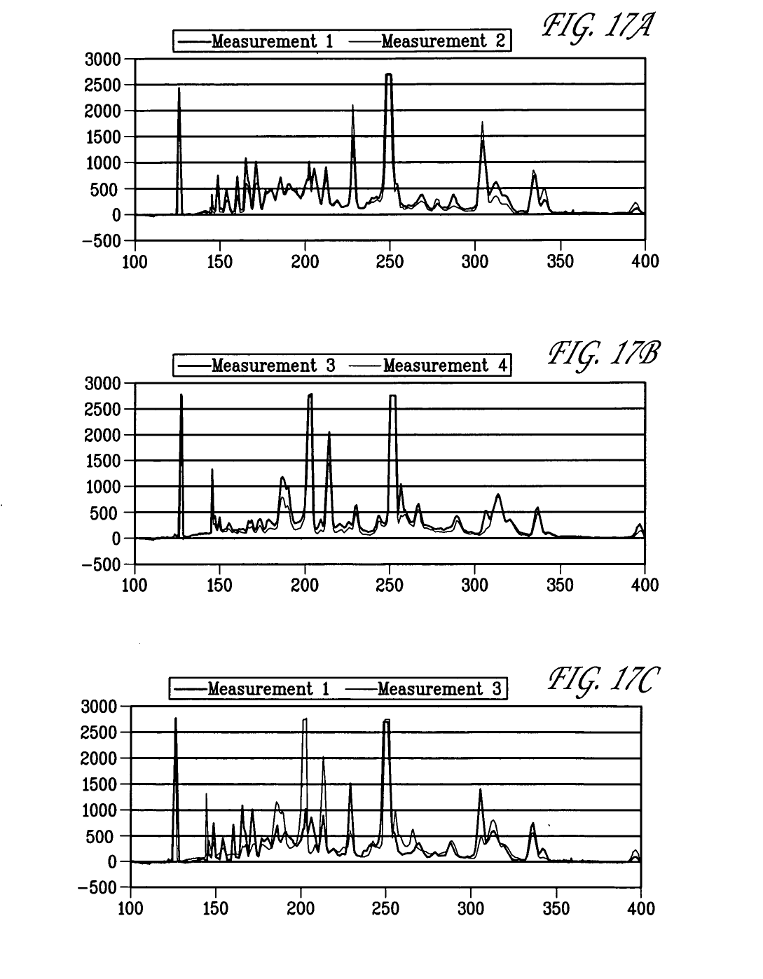

Particular viral species were found to have specific protein fingerprints. As seen in FIG. 17, multiple injections of bacteriophage T2 gave a specific pattern in the generated electropherogram, which was reproducible over run-to-run (FIG. 17A) or day over day. Analysis of bacteriophage T4 also appeared to generate a reproducible signature in the electropherogram (FIG. 17B). Comparing these electropherograms (FIG. 17C), clear differences were apparent. While many of the peaks were similar, the intensity of several of the peaks was different. For example, certain signal peaks appear to be present in one viral preparation but absent in another. These differences indicate that subtle difference exist between viral species of very close origin, which can be detected using the methods and systems of the present invention. Bacteriophage T2 and Bacteriophage T4 appear to differ in the expression level of certain protein species. Different expression levels, where the same apparent protein has different concentrations, were evident by comparing the electropherograms.

Statistical analysis was performed by first splitting the electropherogram into ten individual quadrants. These distinct quadrants were then compared between viral species using a classical least squares (CLS) analysis. CLS analysis of these electropherogram revealed residues greater than 5.3 (for comparing T2 to T4) and 6.9 (for comparing T4 to T2). These differences indicate that the electropherograms were distinguishable.

### EXAMPLE

### Distinguishability of Viral Species

FIGS. 17, A, B and C show the degree to which different viral species can be distinguished and the similarity of measurements for the same species using the systems and processes of the present invention. FIGS. 17A and 17B show baseline and time corrected chromatograms of two measurements for two different species (measurements 1 and 2 are for species X and measurements 3 and 4 are for species Y). FIG. 17C compares measurements for the two species. The retention time correction is based on two calibration standards (HPTS and IgG) and is estimated to be linear (equivalent to invariance of the selectivity parameter). Also, the retention times for the standards are based on a calibration measurement that was used to obtain the dependence of molecular weights on retention time using a set of proteins with known molecular weights, as described further below.

The resulting peak patterns are nearly identical for different measurements of the same species and significantly different for different species. This high degree of reproducibility of peak patterns for a species and the wealth of peak information available enables species identification by use of pattern recognition methods. In contrast to detection of single proteins, which can be misidentified due to small uncorrected shifts in retention times, the information content in the large ensemble of peaks for a single viral species enables detection of these types of species with a reduced likelihood of false alarms, even in the presence of small uncorrected retention time shifts and a small number of unknown contaminant proteins.

It is also possible to use multivariate chemometric methods to analyze mixtures of species that are represented individually in a database. The degree of mixture complexity that can be meaningfully identified by chemometric methods can be determined by spectral analysis methods, for example, the use of orthogonal spectral components of mixtures.

Analysis of various bacteriophage species has indicated vast difference in protein signature between viral agents that are not closely related, supporting the conclusion that viral signatures or fingerprints are adequate for perform identification of unkown viral agents. Distinguishing between closely related viral isoforms is possible using this methodology. In this regard, a library of electropherograms is constructed of the electropherograms of known samples. Unknown samples are identified using the methods and systems of the present invention by comparing the electropherograms of the unknown samples to one or more electropherograms stored in the library.

### EXAMPLE

### A Biotoxin Detection System

A biotoxin detection system was provided according to the present invention. A fully self-contained, portable, hand-held chemical analysis system incorporating “lab on a chip” technologies was developed, also referred herein as the “μChemLab™ system”, or alternatively the “microChemLab system”, or “the system”, or “the device”. The system in this example used microfabricated substrates, i.e., microfluidic chips, to provide fast response times in a low power, compact package. Using microfabrication techniques, parallel separations architecture was provided in which different separation and detection systems were employed simultaneously to provide a separation time fingerprint for each target analyte. By “separation time” as used herein refers to the retention time or the migration time of an analyte undergoing separation.

Samples containing biotoxin species were prepared by diluting samples approximately 1:40 in a sample buffer containing for CGE analysis 5 mM boric acid, 5 mM sodium lauryl sulfate in water drop-wise adjusted to pH 8.5 with 1M NaOH, and for CZE analysis 10 mM phytic acid plus 2 mM DAPS at pH 9.5. Diluted samples were placed on a heating block set at 95° C. for five minutes. After samples were removed from heat, 10 mM fluorescamine dye was added (1 mM working concentration), and samples were vortexed. Fluorescamine labeled biotoxin containing samples (approximately 15 microliters (“μl”)) were then pressure injected into the microfluidic system. After pressure injection of the fluorescamine labeled biotoxin proteins an EK injection was performed followed by a electrophoretic separation of the protein biotoxin analytes. LIF detection was performed using two diode lasers which generated excitation light between 392 and 405 nm and a PMT detector for signal collection.

Based on the molecular weight (CGE analysis) and mass/charge ratio (CZE analysis), biotoxins have a particular migration time. As seen in FIG. 10, during CGE analysis, ricin (a castor bean toxin) migrates to the detection window in approximately 260 seconds which is correlated to the migration of a standard which is approximately 66 kDa, the molecular weight of ricin. CZE has similarly discriminated protein biotoxins, including but not limited to ricin. The pattern of peak migration in CZE analysis is distinguishable from that for CGE analysis. Using these separation techniques in parallel, it is possible to discriminate close isoforms of other toxins, by CZE that could only roughly discriminated with CGE analysis only. These techniques demonstrate that the various methods of the present invention are capable of: 1) detection of the protein biotoxins 2) discriminating between compositionally close, but functionally distinct, toxin isoforms. For other biotoxins, closely related specie isoforms such as S. enterotoxin A & B using two-channel analysis were clearly able to determine the molecular weight and the mass charge ratio which enable the discrimination of these closely related isoforms.

### EXAMPLE

### Analysis of Bacterial Cells and Spores

Cells or spores of bacterial samples were first lysed and the proteins solubilized. Bacterial samples were typically first diluted (approximately 1:40) in a buffer containing 10% SDS at a high pH such as 12. Some bacteria, such as E. coli, do not require such aggressive treatment. Once these sample were dissolved in buffer, they were heated to approximately 100° C. for a period of ten minutes or more. This resulted in, depending on the bacterial species, the lyses and protein solubilization of between 10 and 90% of the viable cells in the sample. A fraction of this sample was then placed in the standard CGE separation buffer containing 5 mM boric acid, 5 mM sodium lauryl sulfate in water drop-wise adjusted to pH 8.5 with 1M NaOH. The proteins were then labeled using an appropriate dye, such as fluorescamine. Labeled samples were then injected on to the microfluidic chip using a gas tight syringe as described above. EK injection and separation was then performed as described above.

### EXAMPLE

### Small Molecule Detection

Small fluorescent molecules or molecules derivatized with a fluorescent molecule were analyzed using this processes and systems described herein. Samples containing molecules having molecular weights of about 500 amu or smaller, such as 10 pM HPTS, were dissolved in a standard buffer used for either CGE or CZE analysis. The samples were then analyzed using similar conditions as described for either CZE or CGE analysis. Analysis of small molecules is not limited to these two techniques, and could be extended for the use of small molecule detection using multiple other separation techniques based on an applied electric filed such as but not limited to MEKC, CEC, HPLC, and native gel electrophoresis.

### EXAMPLE

### Analysis of Eukaryotic Cells and Tissues

Samples of eukaryotic cells and tissues are first lysed and the proteins solubilized. Cell and tissue samples are first solubilized in a phosphate buffer, or an appropriate buffer containing detergent such as SDS, Triton-X or NP-40, at a neutral pH such as 7.4. Once these sample are dissolved in buffer, they are heated to approximately 100° C. for a period of five minutes or more. A fraction of this sample is then placed in the standard CGE separation buffer containing 5 mM boric acid, 5 mM sodium lauryl sulfate in water drop-wise adjusted to pH 8.5 with 1M NaOH for injection onto the microfluidic chip. The proteins are then labeled using an appropriate dye, such as fluorescamine. Labeled samples are then injected on to the microfluidic chip using a specially modified gas tight syringe as previously described. Subsequently an EK injection and separation are then performed as previously described in “Detection of Viral Signatures”.

1. A process for identifying a virus comprising proteins, said process comprising:
solubilizing at least a portion of the proteins of the virus to provide solubilized proteins;
providing a microfluidic chip;
optionally preconcentrating said solubilized proteins on said microfluidic chip;
labeling at least a portion of said solubilized proteins to provide labeled proteins on said microfluidic chip;
electrokinetically injecting at least a portion of said labeled proteins into at least one microchannel electrophoretic separator on said microfluidic chip;
electrophoretically separating at least a portion of said labeled proteins in a constant-current mode to provide separated proteins on said microfluidic chip;
detecting at least a portion of said separated proteins on said microfluidic chip using a detector, wherein said detecting generates signals correlate to the concentration and separation time of said separated proteins; and
analyzing said signals to identify the virus.

2. The process of claim 1, wherein said detector is a laser-induced fluorescence detector.

3. The process of claim 1, wherein said proteins include viral coat proteins.

4. The process of claim 1, wherein the solubilized proteins are labeled with a fluorescent dye.

5. The process of claim 1, wherein said analyzing includes correlating said concentration and said separation time to a viral protein signature.

6. The process of claim 1, wherein said electrophoretically separating includes using at least two parallel electrophoretic separations.

7. The process of claim 6, wherein said at least two parallel electrophoretic analyses individually comprise capillary gel electrophoresis and capillary zone electrophoresis.

8. The process of claim 1, wherein said preconcentrating comprises:
providing a solution comprising solubilized proteins and ions in a first channel residing in said microfluidic chip; and
conducting ions from said first channel through a porous surface to a second channel residing in said microfluidic chip.

9. The process of claim 8, wherein said porous surface comprises a cover material bonded to a rough surface.

10. A process, comprising:
solubilizing components of a sample, said sample comprising a chemical or a biological agent to provide solubilized components;
optionally preconcentrating said solubilized components;
labeling at least a portion of said solubilized components with a fluorescent dye to provide labeled components;
injecting said labeled components electrokinetically into at least one microchannel electrophoretic separator;
separating the labeled components electrophoretically using a controlled electric field, said controlled electric field operating in a constant-current mode;
detecting the separated components with a detector, said detector generating signals, the generated signals being correlated to the concentration and separation time of the labeled components; generating an agent component signature comprising said concentration and said separation time; and
correlating said agent component signature to the identity of the chemical or biological agent.

11. The process of claim 10, wherein said detector is a laser-induced fluorescence detector.

12. The process of claim 10, wherein said preconcentrating comprises:
providing a solution comprising solubilized proteins and ions in a first channel residing in said microfluidic chip; and
conducting ions from said first channel through a porous surface to a second channel residing in said microfluidic chip.

13. The process of claim 12, wherein said porous surface comprises a cover material bonded to a rough surface.

14. The process of claim 7, wherein said biological agent comprises a biotoxin, a bacterium, a virus, a nucleic acid, a portion of biotoxin, a portion of a bacterium, a portion of a virus, a nucleic acid, or any combination thereof.

15. A process, comprising:
solubilizing components of at least two samples comprising a chemical agent, a biological agent, or both, to provide solubilized components;
optionally preconcentrating said solubilized components;
individually labeling the solubilized components with a fluorescent dye;
individually injecting the solubilized components electrokinetically into at least one microchannel electrophoretic separator;
individually electrophoretically separating the labeled components using a controlled electric field operating in a constant-current mode to provide separated components;
individually detecting the separated components with a detector capable of generating signals correlatable to the concentration and separation time of the labeled components;
individually generating an agent component signature comprising said concentration and said separation time; and
identifying a chemical agent or biological agent isoform among the individual agent component signatures.

16. The process of claim 15, wherein said detector is a laser-induced fluorescence detector.

17. The process of claim 15, wherein said biological agent comprises a biotoxin, a bacterium, a virus, a nucleic acid, a portion of biotoxin, a portion of a bacterium, a portion of a virus or any combination thereof.

18. The process of claim 15, wherein said preconcentrating comprises:
providing a solution comprising solubilized proteins and ions in a first channel residing in said microfluidic chip; and
conducting ions from said first channel through a porous surface to a second channel residing in said microfluidic chip.

19. The process of claim 18, wherein said porous surface comprises a cover material bonded to a rough surface.

20. A system, comprising:
a microfluidic chip, comprising;
an injection port for receiving samples comprising protein;
an optional preconcentrator;
an electrokinetic pump for transporting proteins to an electrophoretic microchannel separator, said electrophoretic microchannel separator capable of separating proteins using a controlled electric field, said controlled electric field operating in a constant-current mode;
a detector giving rise to signals correlatable to the concentration and separation time of the separated proteins; and
a data processor for correlating said signals to the protein signatures of known biological samples.

21. The system according to claim 20, wherein said preconcentrator comprises a porous surface in fluid communication between a first channel provided in said microfluidic chip and a second channel provided in said microfluidic chip.

22. The process of claim 21, wherein said porous surface comprises a cover material bonded to a rough surface.

23. The system according to claim 20, further comprising at least one power supply, said power supply capable of generating at least one full-scale stepped voltage in at least 20 milliseconds and capable of measuring at least one current in at least 20 milliseconds.

24. The system according to claim 23, wherein said power supply further comprises an embedded microprocessor capable of measuring an electric current at least once every 100 milliseconds and capable of updating at least one voltage at least once every 100 milliseconds.

25. The system according to claim 24, wherein said embedded microprocessor is capable of measuring an electric current at least once every 50 milliseconds and is capable of updating at least one voltage at least once every 50 milliseconds.

26. The system according to claim 24, wherein said embedded microprocessor is capable of measuring at least ten electric currents at least once every 100 milliseconds and is capable of individually updating at least ten voltages at least once every 100 milliseconds.

27. The system according to claim 24, wherein the embedded microprocessor comprises a current control feedback algorithm and a timer interrupt, said feedback algorithm operating on the updated voltages and the current measurements by operation of a digital-to-analog converter coupled to said timer interrupt.

28. The system according to claim 20, wherein said proteins comprise viral proteins.

29. A process, comprising:
providing a sample comprising macromolecules derived from a biological entity;
solubilizing at least a portion of said macromolecules to provide solubilized macromolecules;
optionally preconcentrating said solubilized macromolecules;
labeling at least a portion of said solubilized macromolecules with a fluorescent dye to provide labeled macromolecules;
electrokinetically injecting at least a portion of said labeled macromolecules into a microchannel electrophoretic separator;
electrophoretically separating said labeled macromolecules using a controlled electric field operating in a constant-current mode to provide separated macromolecules;
detecting said separated macromolecules using a detector capable of generating signals, said signals capable of being correlated to the concentration and separation time of said separated macromolecules;
generating a macromolecular signature, said signature comprising said concentration and macromolecular separation time; and
analyzing said macromolecular signature to identify the biological entity.

30. The process of claim 29, wherein said detector is a laser-induced fluorescence detector.

31. The process of claim 29, wherein said macromolecules include amino acids, nucleic acids, or both.

32. The process of claim 29, wherein said preconcentrating comprises:
providing a solution comprising solubilized proteins and ions in a first channel residing in said microfluidic chip; and
conducting ions from said first channel through a porous surface to a second channel residing in said microfluidic chip.

33. The process of claim 32, wherein said porous surface comprises a cover material bonded to a rough surface.

34. A system, comprising:
an injection port for receiving biological samples comprising biological macromolecules;
a microfluidic chip in fluid communication with said injection port, said microfluidic chip comprising:
an optional preconcentrator in fluid communication with said injection port;
an electrokinetic pump in fluid communication with said injection port capable of transporting said biological macromolecules to an electrophoretic microchannel separator comprising a controlled electric field, said controlled electric field operating in a constant-current mode;
said electrophoretic microchannel separator capable of separating said biological macromolecules;
a detector capable of detecting the presence of said separated biological macromolecules, said detector giving rise to signals being correlatable to the concentration and separation time of said separated biological macromolecules; and
a data processor for correlating said signals to a biological macromolecular signature of a biological entity.

35. The system of claim 34, wherein said preconcentrator comprises a porous surface in fluid communication between a first channel provided in said microfluidic chip and a second channel provided in said microfluidic chip.

36. The process of claim 35, wherein said porous surface comprises a cover material bonded to a rough surface.

37. A system, comprising:
a microfluidic sample injection port capable of receiving a liquid comprising proteomic substances;
a microfluidic chip, comprising:
a preconcentrator in fluidic communication with said injection port, said preconcentrator comprising:
a porous surface in fluid communication between a first channel provided in said microfluidic chip and a second channel provided in said microfluidic chip, wherein the first and second channels comprise deep etched portions in said microfluidic chip and a shallow etched portions in said deep etch portions, said porous surface comprising a cover material bonded to a rough surface, said rough surface being contiguous to said shallow etched portions;
a microchannel capillary zone electrophoresis separator or a microchannel capillary gel electrophoresis separator in fluid communication with said preconcentrator; and
a detector capable of detecting the presence of proteomic substances, said detector capable of generating signals correlatable to the concentration and separation time of said proteomic substances.

38. The system of claim 37, further comprising a data processor for receiving said signals and generating a proteomic signature of a biological entity.

39. The system of claim 38, wherein the data processor is contained within the housing of the system.

40. The system of claim 39, further comprising an information display coupled to said data processor.

41. A system, comprising:
at least one separation module, comprising:
a microfluidic sample injection port capable of receiving a liquid under pressure, said liquid comprising proteomic substances;
a fluidic system capable of electrokinetically transporting said liquid and capable of separating said proteomic substances by molecular size; and
a microfluidic fluorescence detector capable of detecting the concentration and separation time of said proteomic substances; and
a power supply capable of monitoring and controlling electric currents and voltages of said fluidic system, said power supply capable of generating at least one full-scale stepped voltage in at least 20 milliseconds and capable of measuring at least one current in at least 20 milliseconds.

42. The system according to claim 41, wherein said power supply further comprises a microprocessor capable of measuring an electric current at least once every 100 milliseconds and capable of updating at least one voltage at least once every 100 milliseconds.

43. The system according to claim 41, wherein said embedded microprocessor is capable of measuring an electric current at least once every 50 milliseconds and is capable of updating at least one voltage at least once every 50 milliseconds.

44. The system according to claim 41, wherein said embedded microprocessor is capable of measuring at least ten electric currents at least once every 100 milliseconds and is capable of individually updating at least ten voltages at least once every 100 milliseconds.

45. The system according to claim 42, wherein said microprocessor comprises a current control feedback algorithm and a timer interrupt, said feedback algorithm operating on the updated voltages and the current measurements by operation of a digital-to-analog converter coupled to said timer interrupt.

46. The system of claim 41, further comprising a power source.

47. The system of claim 46, further comprising a housing enclosing said separation module and said power supply, said power source being located within said housing or external to said housing.

48. The system of claim 47, wherein said power source includes a battery, a fuel cell, or both, said power source being located within said housing.

49. The system of claim 41, wherein said fluidic system is capable of separating the proteomic substances using capillary gel electrophoresis or capillary zone electrophoresis.

50. The system of claim 41, wherein said fluidic system comprises a microfluidic chip for electrokinetically transporting said liquid, said microfluidic chip comprising a separation channel for separating said proteomic substances and a preconcentrator.
